(19) United States
(12) Patent Application Publication
(10) Pub. No.: US 2023/0125132 A1
(43) Pub. Date: Apr. 27, 2023

(54) Clinical Pharmacy Management System

(71) Applicant: Mobile MediClaim, Inc., Lindenhurst, IL (US)

(72) Inventor: Jean Jendusa, Lindenhurst, IL (US)

(21) Appl. No.: 17/817,291

(22) Filed: Aug. 3, 2022

Related U.S. Application Data
(60) Provisional application No. 63/228,770, filed on Aug. 3, 2021.

(51) Int. Cl. G16H 40/67; G16H 15/00; G16H 40/20; G06Q 40/08

(52) U.S. Cl. CPC G16H 40/67; G16H 15/00; G16H 40/20; G06Q 40/08

(57) ABSTRACT

A cloud-based medical system for use by clinical pharmacists in the areas of electronic health record (EHR) management, medical credentialing, billing, access to consulting specialists, and patient tracking.

### RELATED APPLICATION

The present disclosure claims the filing priority of U.S. Provisional Application No. 63/228,770, titled “Clinical Pharmacy Management System” and filed on Aug. 3, 2021. The '770 patent application is hereby incorporated by reference.

### TECHNICAL FIELD OF THE INVENTION

The present application relates to cloud-based services for use by clinical pharmacists in the areas of electronic health record (EHR) management, medical credentialing, billing, access to consulting specialists, and patient tracking. Particularly, the application relates to cloud-based systems as well as methods and devices for creating such systems and hereby incorporates by reference the Mobile Mediclaim website (i.e., https://mobilemediclaim.com) as if fully set forth herein. Also incorporated by reference to the subject application are the following publications: US. Published Application Nos. US2011/0282689 to Patel, US2017/0262614 to Vishnubhatla, and US2019/0385733 to Kaye.

### BACKGROUND OF THE INVENTION

Typically, medical claims are bills which are sent to health insurance companies in order to receive payment for any service or product, including pharmaceuticals and medical supplies, that a healthcare professional used to render care. A specific medical code is used to describe the type of service or product provided and can include a diagnosis, medical procedure, medical supplies, use of medical equipment, pharmaceuticals, and medical transportation among other things. Providers include all medical codes related to a patient's encounter and the insurance company analyzes the medical codes to determine how to provide reimbursement.

Previously, these claims for reimbursement had been made by filling out a “manual claim”. A manual claim is a paper form that is sent to the payer through the mail. Because of advancements in technology, healthcare professionals can now make paperless “electronic claims”. Electronic claims have been further optimized with the use of cloud-based technology, or cloud computing. Specifically in the pharmaceutical field, Pharmacists would be required to submit medical claims first through a physician's office which would then forward the claim on to the appropriate payer.

Cloud computing is the practice of using a network of remote servers hosted on the internet to store, manage, and process data. The premise of cloud computing relies on sharing resources to achieve coherence and economies of scale. Access to a cloud can be limited to a certain organization, or it can be larger and available to the public.

In the present application, the cloud-based system allows for, among other things, medical professionals, and pharmacists to care for their patients anywhere from almost anywhere. The system can be used by clinical pharmacists in the areas of electronic health record (EHR) management, medical credentialing, billing, access to consulting specialists, and patient tracking. Further, in an embodiment, users (i.e., medical professionals) are able to document their encounters and bill for their services immediately at the point of care. Pharmacists specifically, are able to bypass a third-party physician's office and submit claims directly to the payer. This immediacy has never before been available. Even through the use of traditional “electronic claims” sent through a physician's offices, the time for receiving a response is still significant. With the system and method of the present disclosure much of the payer information can be available through the cloud thereby eliminating wasted money, extra steps, and longer response times.

These and other problems are addressed in the present disclosure to provide a system and methods with numerous advantages in operation and effectiveness.

### BRIEF DESCRIPTION OF THE DRAWINGS

For the purpose of facilitating an understanding of the subject matter sought to be protected, there are illustrated in the accompanying drawings embodiments thereof, from an inspection of which, when considered in connection with the following description, the subject matter sought to be protected, its construction and operation, and many of its advantages should be readily understood and appreciated.

FIG. 1A is a schematic showing communication between the various entities involved in a medical professional and patient billing scenario;

FIGS. 1B and 1C are portions of screen shots of a patient appointment interface which allows a user to choose a date, time and “appointment type” (service) that is desired as well as a provider for such services;

FIG. 2 is portions of a screen shot showing how a user is allowed to search/filter appointments and change the status of appointments—e.g., booked, confirmed, arrived, completed, cancelled, or billed;

FIG. 3A is portions of a screen shot showing how a user can set parameters for appointment types;

FIG. 3B is a screen shot showing the ability of the system to create and add appointment types;

FIG. 4 is portions of a screen shot which displays the ability to archive claims and search archived claims;

FIG. 5 is portions of a screen shot showing a “Manage Patient” interface which allows users to, among other tasks, map inbound HL7 CCDA documents to the respective patients;

FIG. 6 is portions of a screen shot showing a “Chart Review” interface which allows supervising doctors to review completed patient charts at, for example, clinics under their supervision;

FIG. 7 is portions of a screen shot showing a “Check Eligibility” interface which allows users to request patient insurance details directly from a carrier;

FIG. 8 is portions of a screen shot showing a “Medical Claims” interface which allows users to view any active claims;

FIG. 9 is a screen shot showing a “Bill A Claim” template under the “Claim Management” interface which allows users to process and submit a claim directly to a carrier;

FIG. 10 is a portion of a screen shot showing an “Additional Information” template under the “Claim Management” interface which allows users to enter additional information for any claim being submitted;

FIG. 11 is portions of a screen shot showing an ERA interface and template which allows a user to see pending, posted, and denied ERAs;

FIG. 12 is portions of screen shots illustrating the template for manually posting ERAs to a patient file;

FIG. 13 is portions of a screen shot showing a “Billing & Payment” interface which allows users to view/search any invoices that have been marked as complete;

FIG. 14 is portions of a screen shot showing a “Payments” interface embodiment and an “Add Payment” template for the system which allow a user to add payment details and see a list of payments made for a patient;

FIG. 15 is a portion of a screen shot showing a “Support Inbox” interface which allows users to contact support personnel directly;

FIG. 16A is portions of a screen shot showing a first embodiment of a “Dashboard” interface for a user which displays details including “total patients”, “total claims”, and “total approved claims”, as well as year-to-date, month-to-date, and week-to-date statistics;

FIG. 16B is portions of a screen shot showing another embodiment of a “Dashboard” interface for a user as well as a template for creating and saving tasks;

FIG. 17 is portions of a screen shot showing a “Doctor Search” interface which allows physician searches;

FIG. 18 is portions of a screen shot showing a “Holiday Setup” interface which allows users to set holidays for facilities;

FIG. 19 is a screen shot of a “Invoice” interface under the “Billing & Payment” heading which allows users to view, edit, and complete invoices for appointments;

FIG. 20 is portions of a screen shot showing a “Knowledge Base” interface which gives users access to a list of topics and a knowledge base library;

FIG. 21 is portions of a screen shot showing timestamped entries for each action in the system;

FIG. 22 is a screen shot of a user login interface;

FIG. 23 is portions of a screen shot showing a “Manage Patient” interface which allows users to view, modify, or remove patients from a patient list;

FIG. 24 is portions of a screen shot showing a summary of personal and medical data collected for a specific patient;

FIG. 25 is a portion of a screen shot showing an interface which allows a user to import/export CCDA data received via HL7 with existing patient data in the system;

FIG. 26 is a portion of a screen shot showing an interface which allows a user to view appointment histories;

FIG. 27 is a portion of a screen shot showing an interface which allows a user to create and view education resources;

FIG. 28 is a screen shot showing an interface which allows a user to view and modify patient information;

FIG. 29 is a screen shot showing an interface which allows a user to view and enter insurance details for a patient;

FIG. 30 is a partial screen shot showing upcoming appointments scheduled for a specific patient;

FIG. 31 is portions of a screen shot showing an interface which allows a user to upload patient files to the system;

FIG. 32 is a partial screen shot showing “Claim History” associated with a specific patient;

FIG. 33 is a partial screen shot showing “Invoices” associated with a specific patient;

FIG. 34 is a partial screen shot showing an interface which allows a user to upload patient data in .XML or .CSV;

FIG. 35A is portions of a screen shot showing a “Report” interface which allows a user to generate reports based on data in an account;

FIG. 35B is a partial screen shot of a “Support Inbox” interface which allows a user to communicate with a support staff;

FIG. 36 is portions of a screen shot showing a “Prescription renewal” interface which allows a user to review prescription renewal requests;

FIG. 37 is portions of a screen shot showing an “Appointment” interface including a “Scheduler Setup” which allows a user to view, create and modify weekly schedules for providers in a facility;

FIG. 38 is portions of a screen shot showing an interface which allows a user to capture relevant medical information regarding a patient while conducting an appointment;

FIG. 39 is a screen shot showing an interface which allows a user to enter information pertaining to the Subjective, Objective, Assessment, and Plan (aka SOAP note);

FIG. 40 is a screen shot showing an input box which allows a user to validate and sign SOAP details and superimpose a signature onto the details once the appointment is complete;

FIG. 41A is portions of a screen shot showing a first embodiment of a “SOAP Template” interface which allows a user to create custom templates to be used in SOAP note creation for appointments, etc.;

FIG. 41B is portions of a screen shot showing a second embodiment of a “SOAP Template” interface which allows a user to create custom templates to be used in SOAP note creation for appointments, etc.;

FIG. 42 is portions of a screen shot showing a “Stripe Configuration” interface which allows a user to configure a Stripe account for electronic processing and payment of invoices; and

FIG. 43 is portions of a screen shot showing a “Billing & Payment” interface which allows a user to review Electronic Remittance Advice (ERA) documents that have been received from insurance carriers in response to claims.

### DETAILED DESCRIPTION OF PREFERRED EMBODIMENTS

While this invention is susceptible of embodiments in many different forms, there is shown in the drawings and will herein be described in detail, a preferred embodiment of the invention with the understanding that the present disclosure is to be considered as an exemplification of the principles of the invention and is not intended to limit the broad aspect of the invention to any embodiments illustrated.

The present system and methods, flow diagrams and components of which are illustrated in the appended figures, provide services for use by clinical pharmacists in the areas of Electronic Health Record (EHR) Management, medical credentialing, billing, access to consulting specialists, and patient tracking. In at least one preferred embodiment, the system and methods provide the ability for healthcare professionals, specifically pharmacists, to:

i. instantly know the medical insurance coverage available to a patient,

ii. connect with other healthcare professionals,

iii. print and send documents from the system nearly anywhere, and

iv. potentially connect with all medical insurance payers in the country.

Because healthcare information is incredibly sensitive, the present system and methods also contemplate the need for an extra layer of security by utilizing multi-factor authentication. The transmission of a single-use security code provides login verification and device authentication. The security code would only be valid for a single login session and only for a defined period of time, as is known in the art.

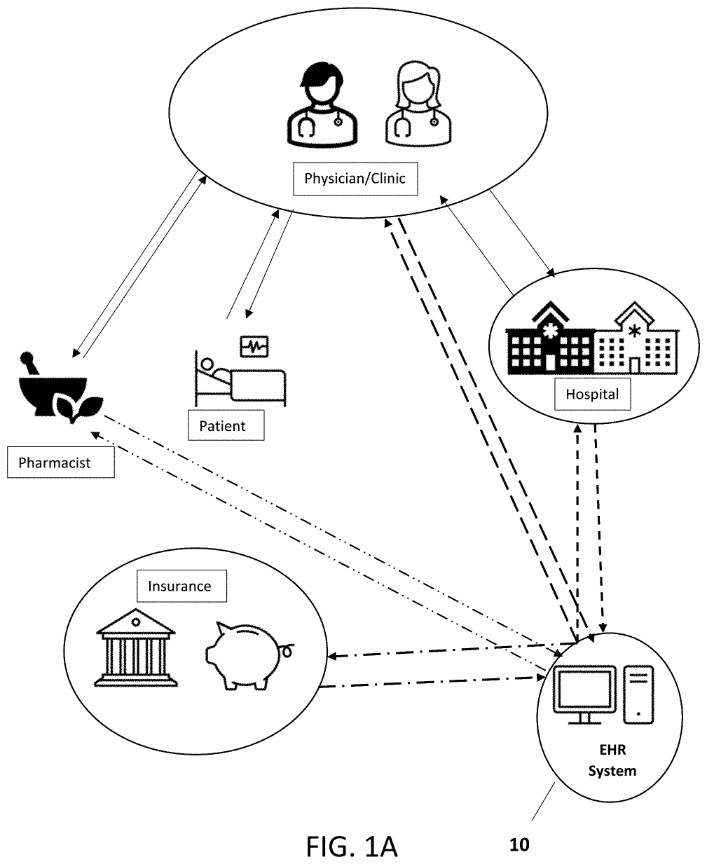

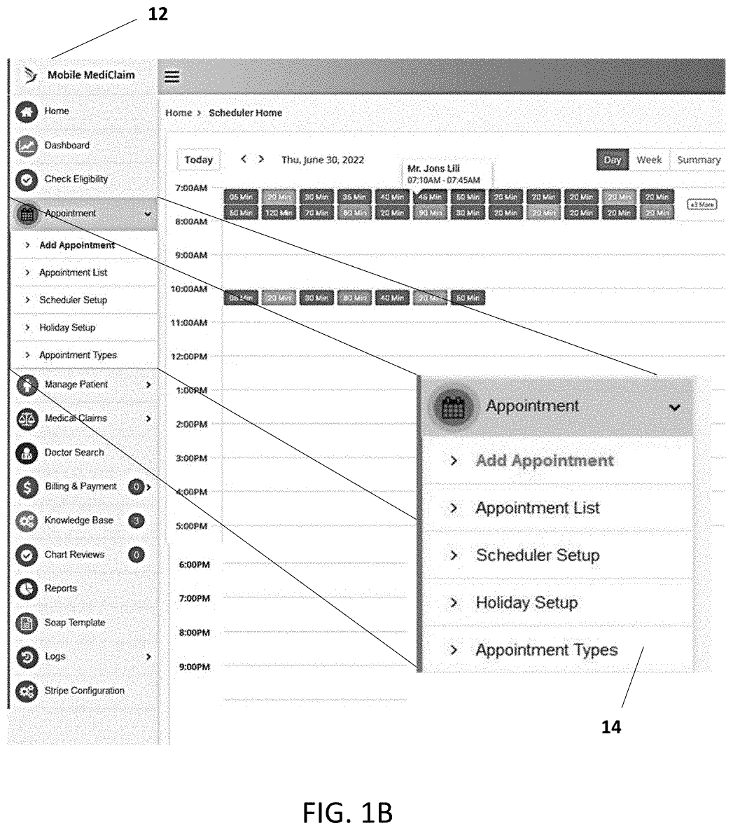

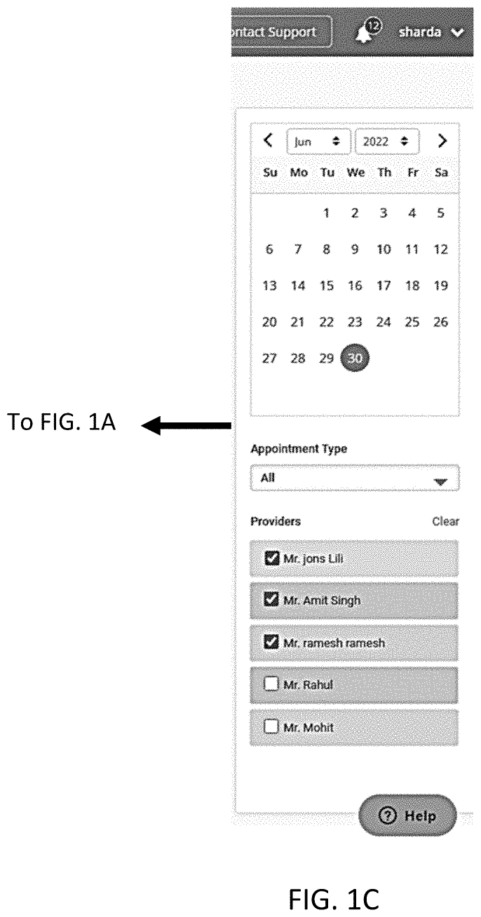

The present disclosure also describes a system and methods which allow pharmacists to network with other pharmacists and physicians and actively engage in helping in the treatment and care of patients. The system may be configured to provide the capability to verify pharmacist credentials to certify provider status and receive reimbursement for professional services directly. Ultimately, the system would authorize pharmacists to bill at the point of service, making them non-dependent on a physician's billing office to submit claims, nor requiring any follow-up with them. The disclosed system would ensure seamless integration between dispensing care and medical billing platforms. As shown in FIG. 1A, the system would also provide connectivity directly to all Medical Payer and Third-Party Administrators, in addition to doctors and hospitals. This helps reduce medical and pharmacy spending.

One of the major benefits of the disclosed system is that the cloud-based application is geared toward the clinical Doctor of Pharmacy and, therefore, has information in a database on all the drugs and supplies. This permits a user to pull up details on size, color, etc., for every device and pharmaceutical in the system and use that information in an educational dialogue with patients. Further, the system would provide the ability to pull up and display past prescriptions filled on-line through a PBM for a period of time, preferably for at least the last 12 months. This provides important information and insight to the patient and to the clinical pharmacist, who is able to monitor all possible chemical interactions with all the medications in the patient's body causing changes both positive and negative with the patient. The system would also allow the pharmacist to write prescriptions online and send them to various pharmacies. All of these capabilities are done via the Internet in a HIPPA compliant manner, giving flexibility to the pharmacist who is then able to treat a patient regardless of location.

Referring now to FIGS. 1-43, there is illustrated at least one preferred embodiment of a clinical pharmacy management system, generally designated with the numeral 10. The system 10 is comprised of a cloud-based application, the features, and benefits of which are best described with reference to embodiments of a proposed user-interface as illustrated by the numerous screen displays—i.e., screen shots. These screen shots not only show preferred features and options of the overall system 10 but also demonstrate methods which allow users to check patient benefit eligibility, manage patients, manage claims, view and run diagnostic reports, check and manage administrative matters, and sign in and out.

As shown in numerous of the appended drawing figures, there is disclosed a menu panel 20 having numerous options for selection, some of which open additional sub-menu options, as will be described and illustrated herein. These menu options are selected via a keyboard/mouse interface or using a touchscreen, each of which is well-known in the art.

Beginning with FIGS. 1B and 1C, the “Appointment” menu option is selected, and a drop-down sub-menu is revealed. The sub-menu includes the options of “add appointment”, “appointment list”, “scheduler setup”, “holiday setup”, and “appointment types.” The illustrated screen allows a user to schedule appointments for patients by entering information at the desired time and date. Note left tab “Add Appointment” 12 on interface screen. The user can designate not only the day and time, but an “appointment type” (e.g., service) that is desired as well as a provider for such services (FIG. 1C). As shown, appointment settings allow double and triple bookings to occur for overlap in a clinician's schedule. Finally, a “Help” button appears on most screen options and selection of this option will take the user to a support page or the like.

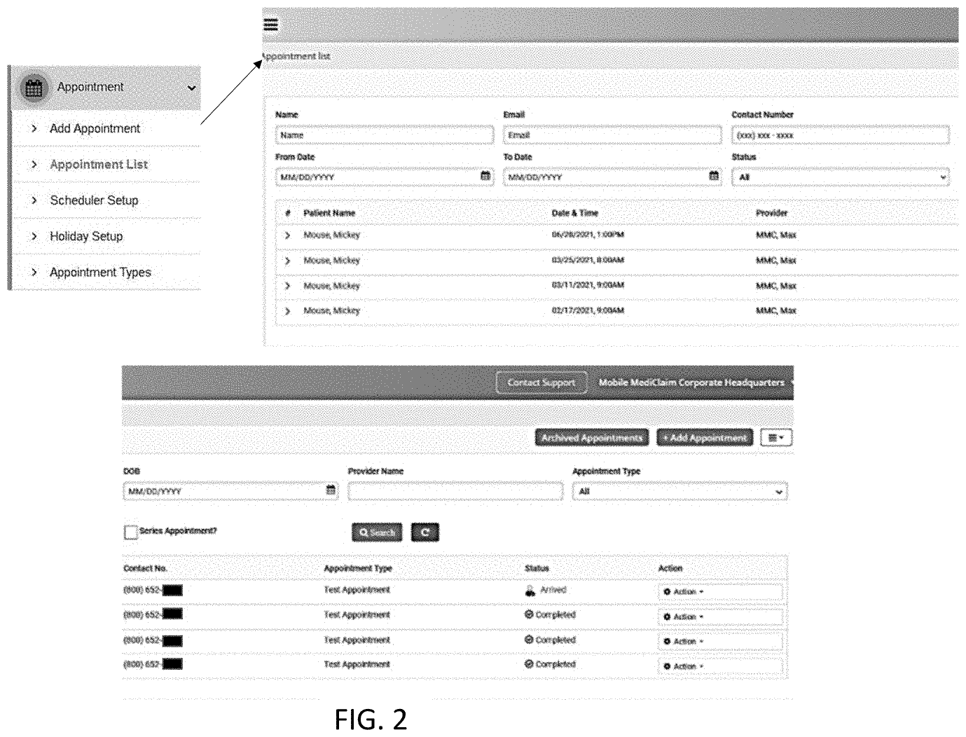

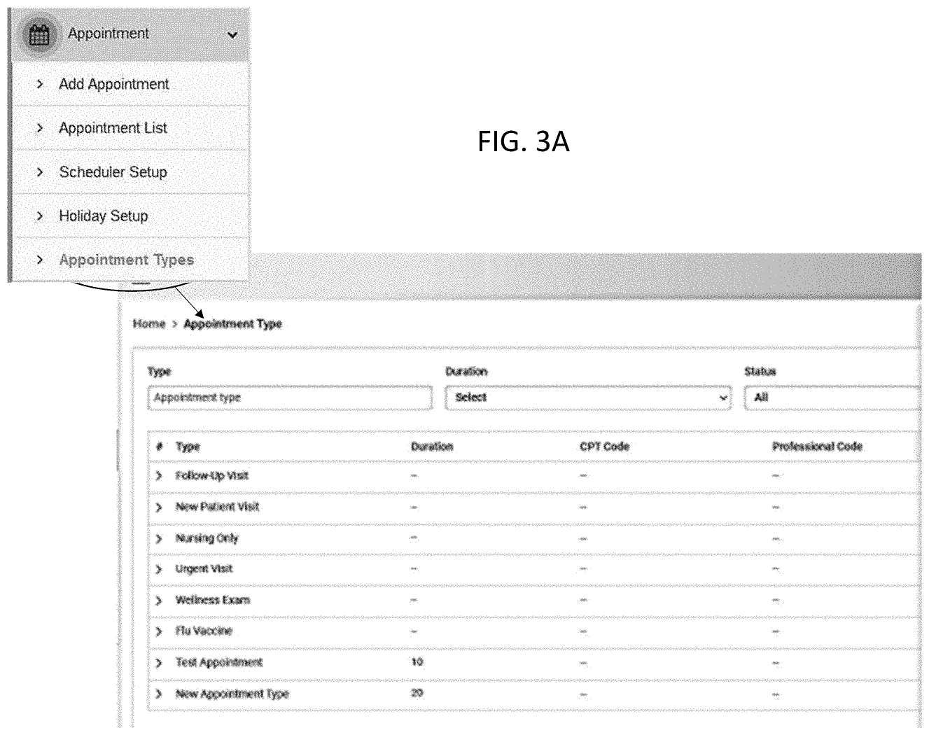

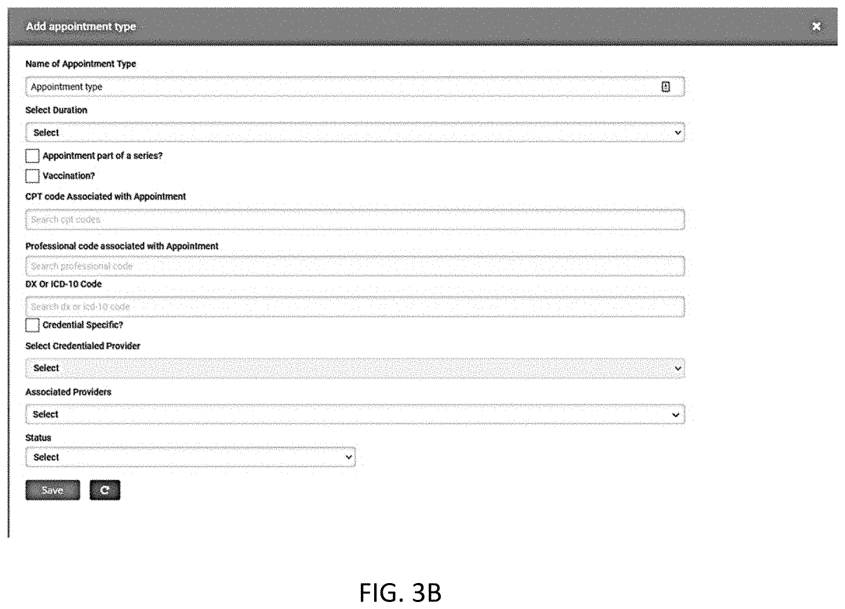

FIG. 2 is another “Appointment” interface screen which allows a user to see an “Appointment List” as well as search/filter, add appointments or change the status of existing appointments—e.g., booked, confirmed, arrived, completed, cancelled, or billed. Once an appointment status is designated as “completed” it is archived. FIGS. 3A and 3B show portions of a screen shot where a user can set parameters for appointment types, including creating new appointment types. Duration, CPT Code and billing codes can be automatically associated with each appointment type to save on input mistakes.

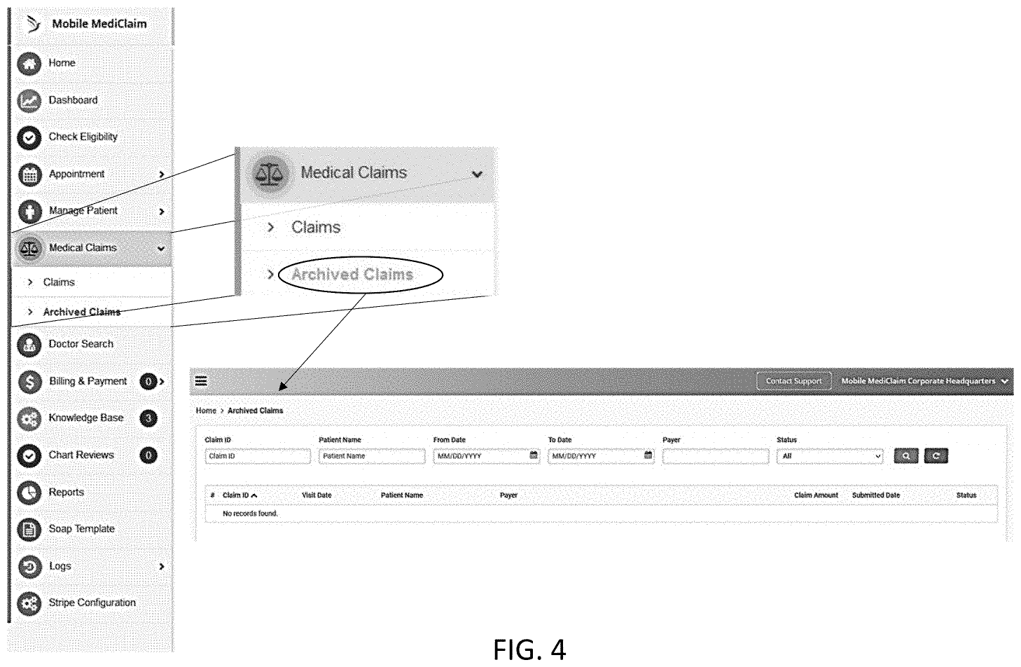

The screen shot of FIG. 4 displays archived claims as part of a submenu under the “Medical Claims” interface. A user is able to search or filter the archived claims by Claim ID, patient name, payer, status or even within a date range.

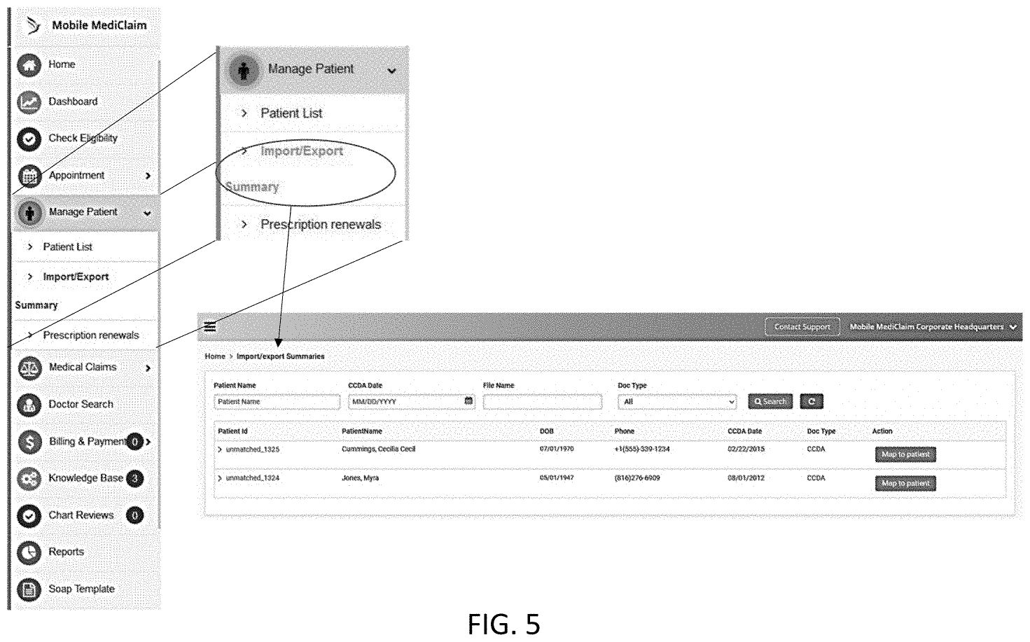

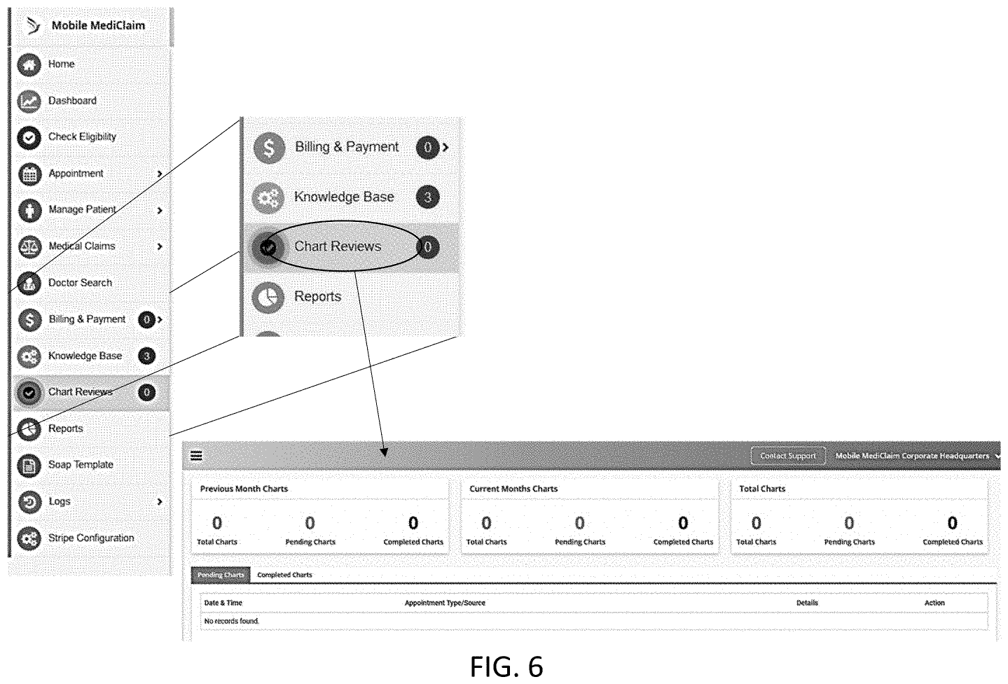

FIG. 5 shows portions of a screen shot of submenu option “Import/Export Summary” under the “Manage Patient” interface. This option allows a user to map inbound HL7 CCDA documents to the respective patients. FIG. 6 is portions of a screen shot showing the “Chart Reviews” interface which allows users to see a summary of “Previous month charts”, “Current Months charts”, and “Total charts” at a glance, while supervising doctors can review completed patient charts for clinics they oversee. Current “pending” and “completed” charts can be listed under the corresponding tabs. A doctor can reject or approve the chart as well as leave notes.

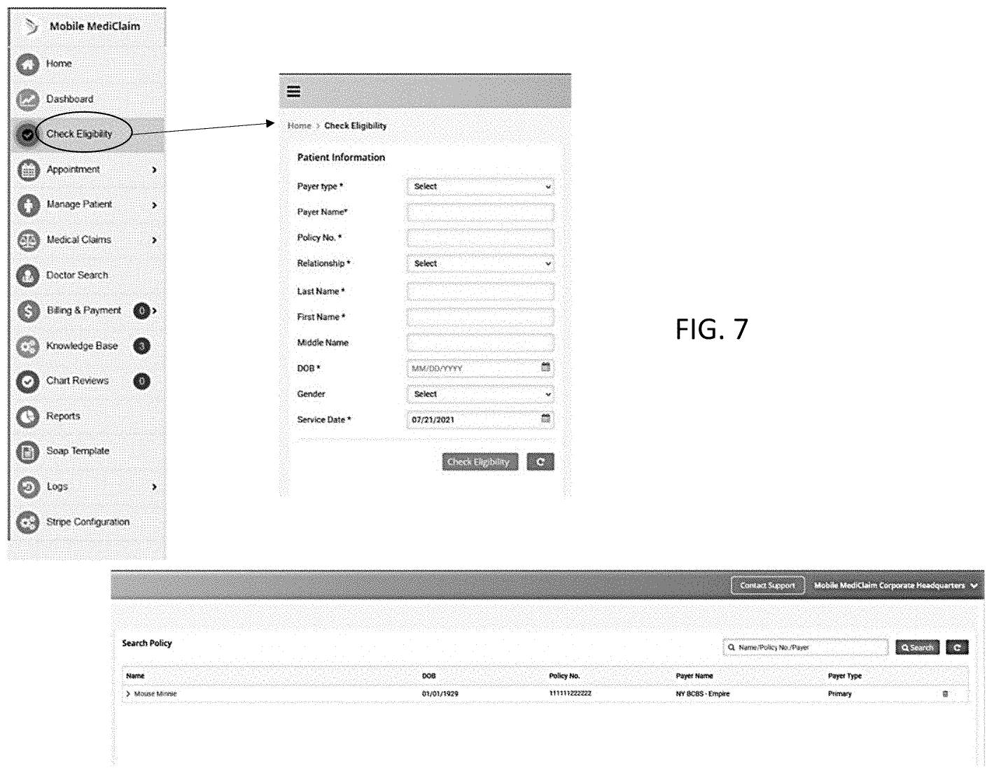

FIG. 7 is portions of a screen shot showing the “Check Eligibility” interface which allows users to request patient insurance details directly from an insurance carrier. Responses are provided in “live-time” (or real-time), meaning all information is current and up-to-date. Search capability is provided as well, using patient name, policy no., or payer.

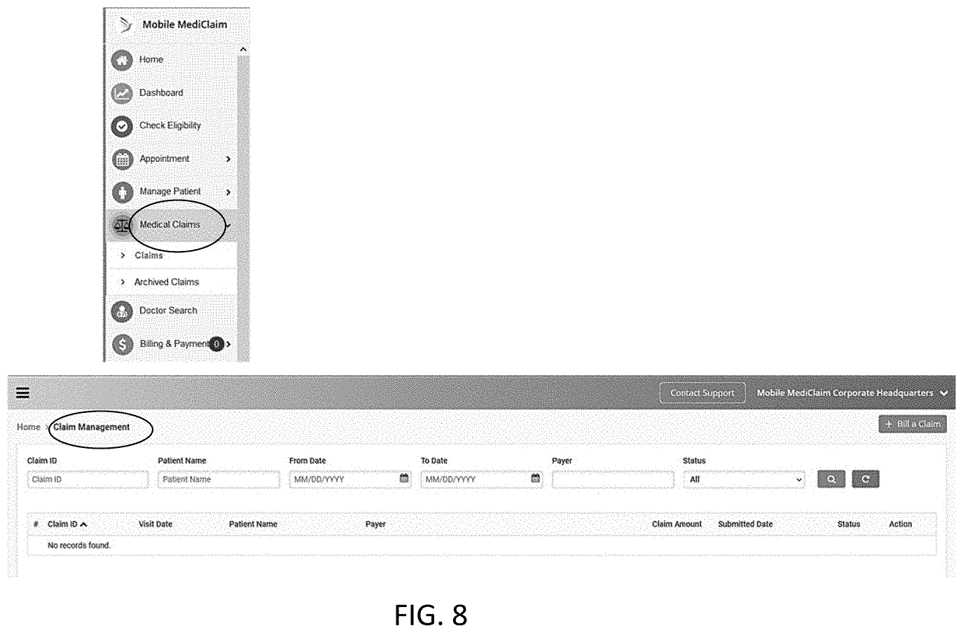

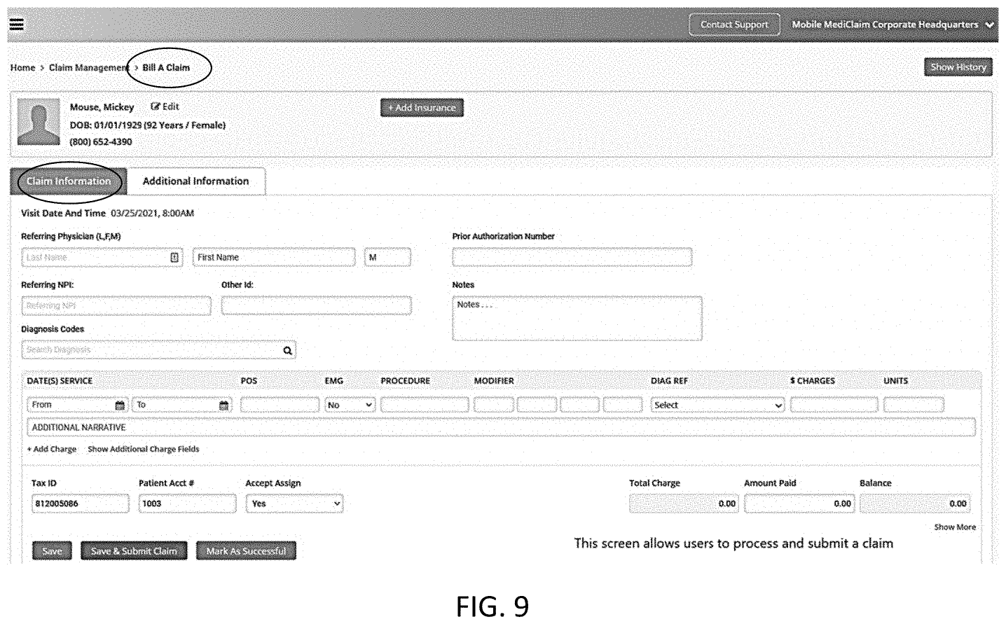

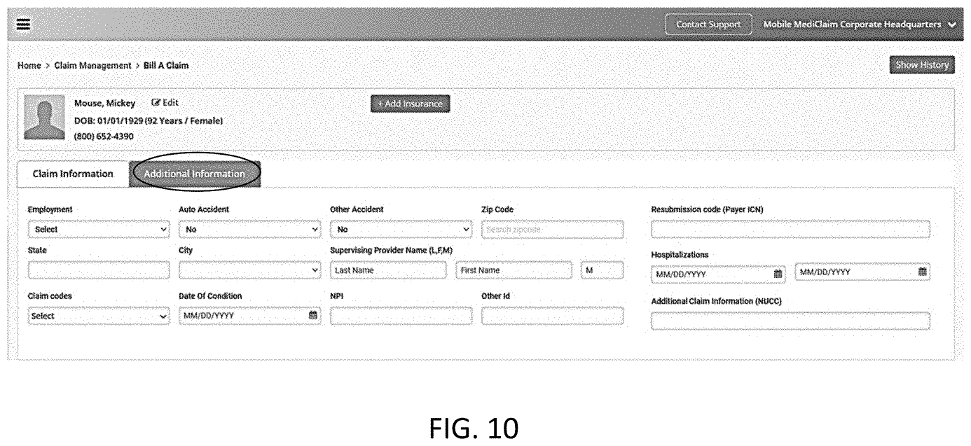

FIG. 8 is portions of a screen shot showing the “Claim management” option under the “Medical Claims” interface. In the “management” screen, the user is allowed to view any active medical claims in the system. By selecting “Bill a Claim” in the upper right corner of the screen, the system allows a user to create a claim by selecting a completed appointment from the list. This takes the user to the screen of FIG. 9 showing an interface which allows users to process and submit a claim. The screen lists all the pertinent information and insurance carrier, including visit date/time, physician, codes, total charge, and the like. Further, under the “Claim management” option, FIG. 10 shows a screen shot of an interface which allows users to enter additional information for any claim being submitted.

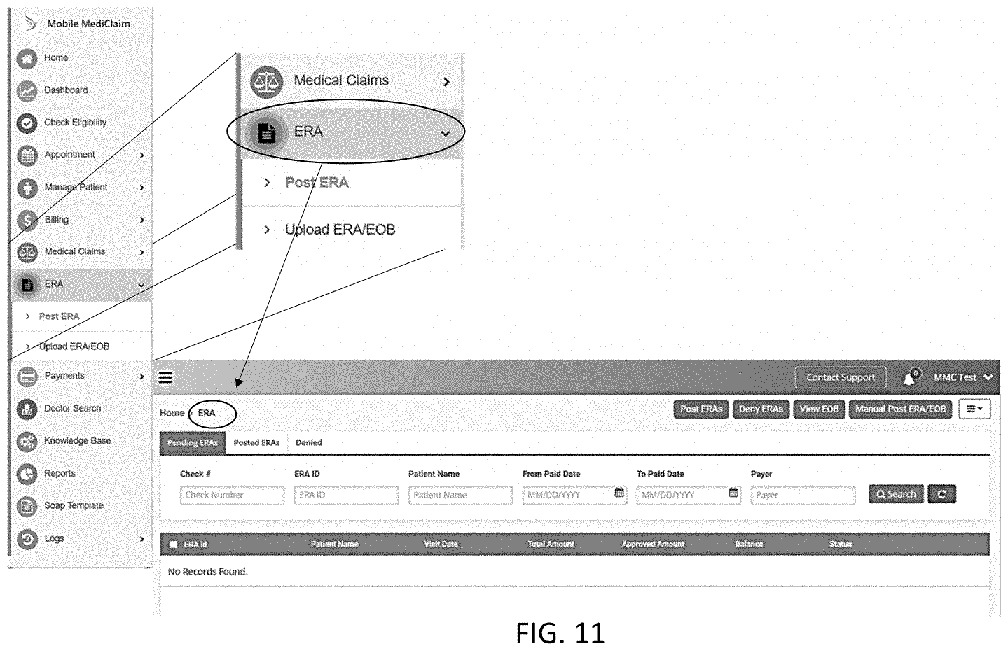

FIG. 11 is portions of a screen shot of one specific embodiment of the system 10 showing a primary menu including a Electronic Remittance Advice or “ERA” interface. The relevant screen allows a user to search and see pending, posted, and denied ERAs, with submenu options to post ERAs, deny ERAs, view EOB, as well as a manual post ERA/EOB. The Electronic Remittance Advice capabilities are fully integrated with Clearinghouse mirrors that reflect back into the system EHR.

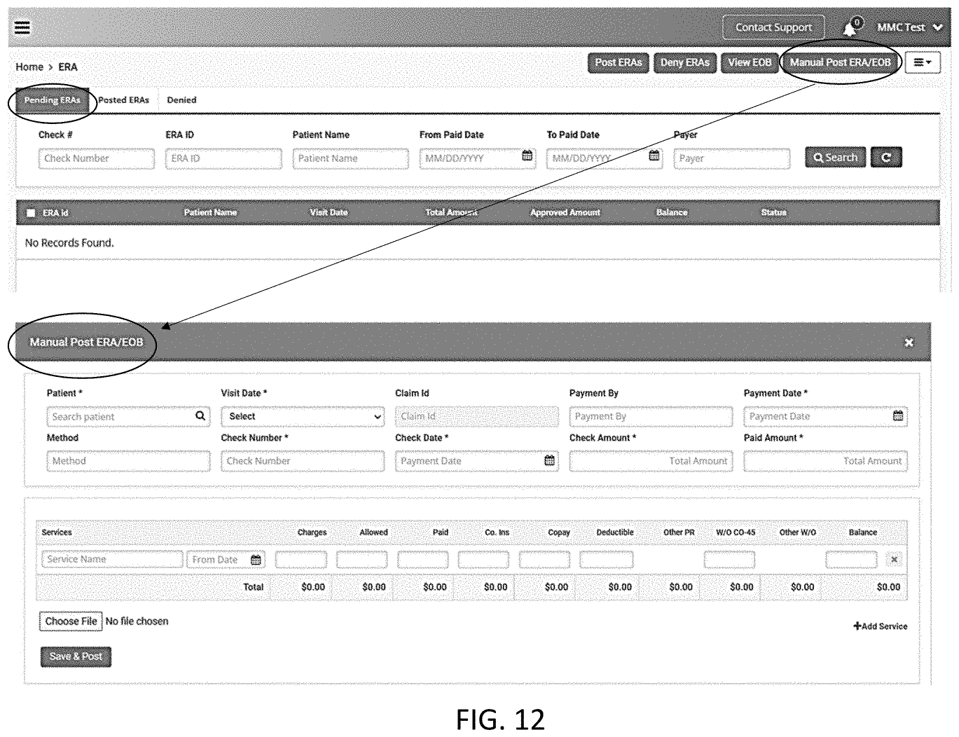

FIG. 12 shows portions of screen shots for viewing and inputting ERAs. Each patient chart shows the detailed claim management for each encounter per clinician. The medical claim shows all the messages from the payor attached and the payment amounts. The system shows the denied ERA's and allows the viewing of the Explanation of Benefits. This provides insight into what the payor is stating to the patient. The system has the ability to do manual posting to the medical claims in the patient file. Manual posting allows for the ability to accurately reflect the claim when a paper check has been mailed to the clinician or patient. The biller has the ability to make the encounter exactly corrected with write off abilities as well. Also, this option allows the biller to create an invoice to the patient with reason codes attached which represent how the payor considered the claim. These features facilitate all aspects of viewing the claims, posting claims and writing off portions of the claim to make the bookkeeping accurate.

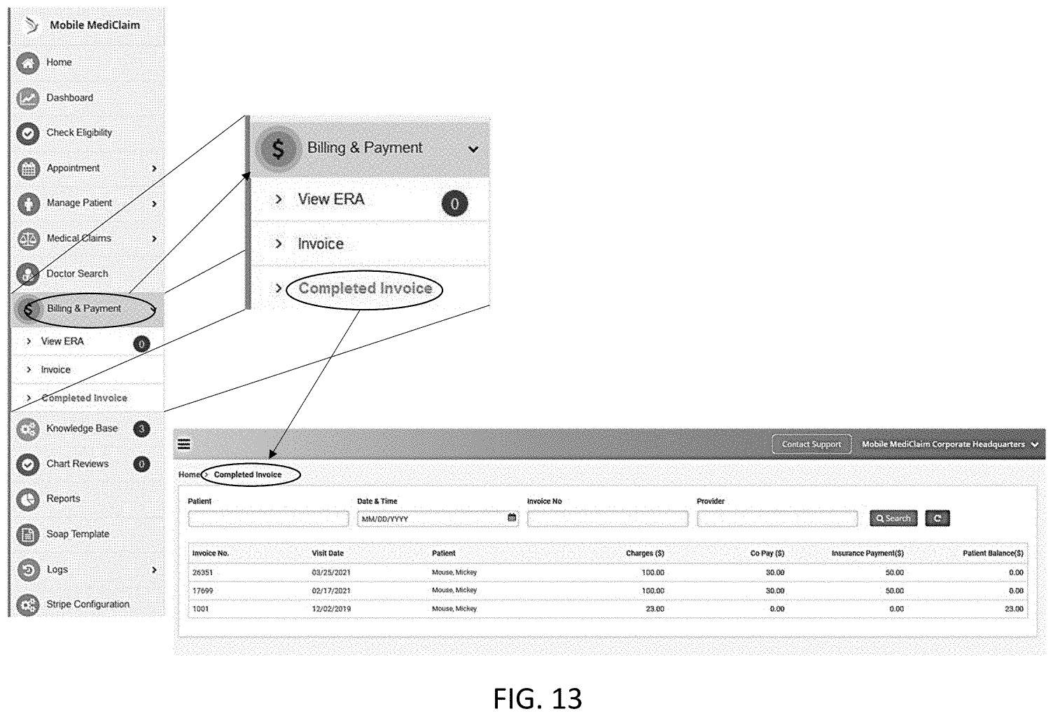

FIG. 13 is portions of a screen shot showing the “Completed Invoices” option under the “Billing & Payment” interface which allows users to view/search any invoices that have been marked as complete. Searches or filters can be performed using patent name, invoice no., provider, or a date range. This particular interface menu is an alternative to that in at least FIGS. 11 and 12, as evidenced by the submenu of “View ERA” in FIG. 13.

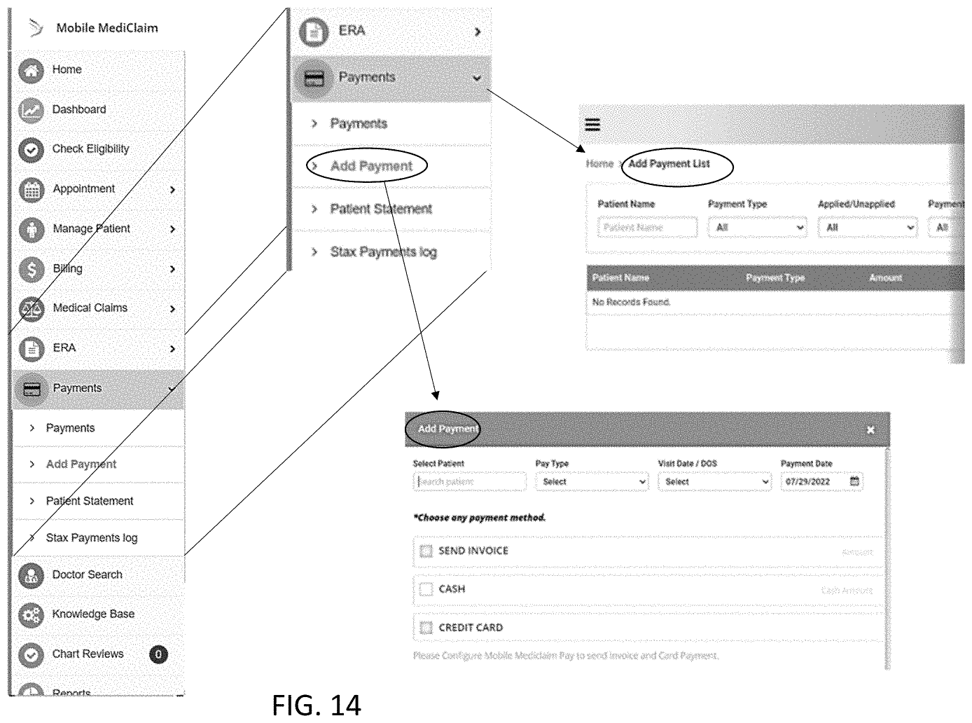

FIG. 14 is multiple portions of screen shots from the “Payments” interface menu. The EHR system is preferably integrated with a merchant processing company. This allows the clinic to collect the copay at the point of sale and process the credit card or cash payment to the claim about to be submitted. There is normally always a copay due at the point of service, so this feature makes it easier to track the finances of the medical claim from the original date of service whereby a copay is collected with cash or credit. The system allows the biller to send detailed invoices out to patients on behalf of the clinics. Paper statements and/or emailed invoices can be sent with payments due to the clinics.

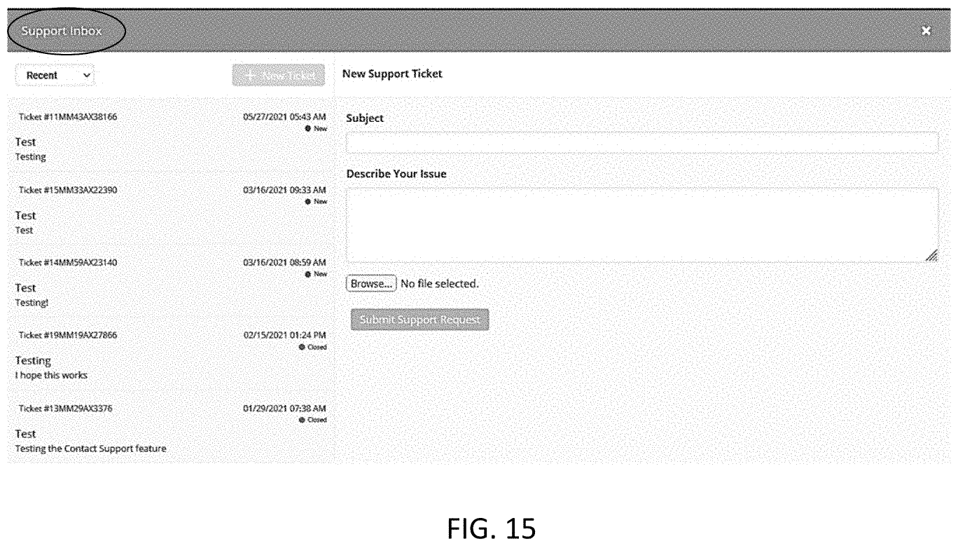

FIG. 15 is a screen shot showing an “Support Inbox” interface which allows users to contact support personnel directly by creating support tickets. A listing of support tickets are shown with a date and status (e.g., new or closed). Any response from the support personnel/staff appears in the support inbox.

Support Ticketing is a unique benefit feature of the EHR system. For example, a clinic now is able to create a support ticket from the EHR to communicate with MMC involving anything related to the system. This includes Education Information, any question pertaining to how a claim was paid or denied, or why a claim is still pending. This allows efficiency with communicating directly from customer service into billing department.

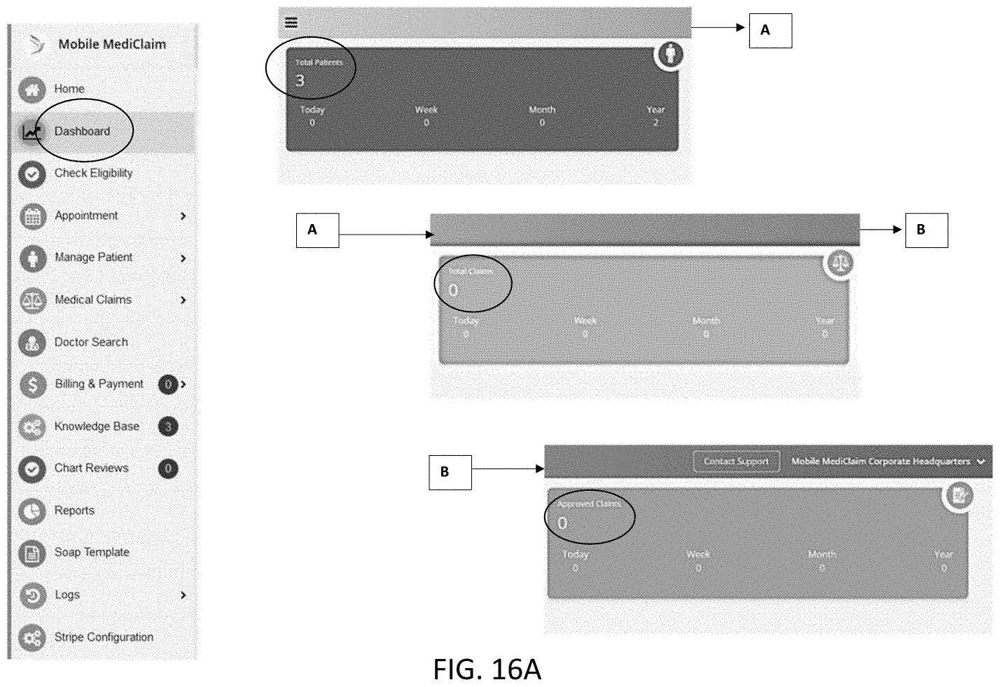

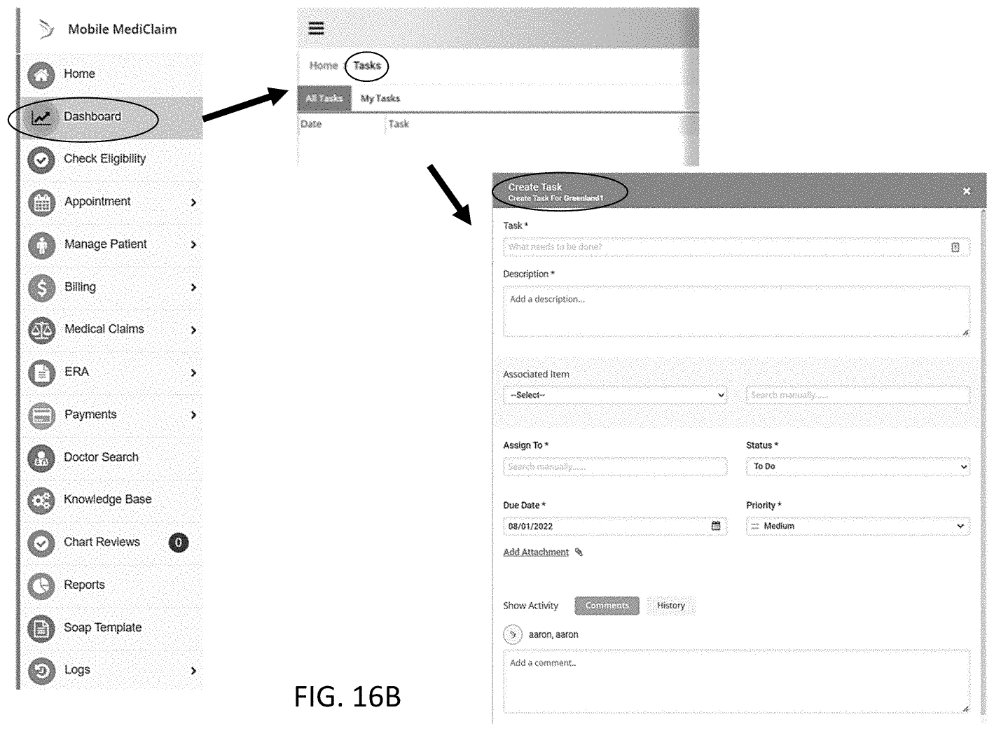

FIG. 16A is portions of a screen shot showing details of a specific embodiment of the “Dashboard” interface of system 10 (see embodiment of FIG. 13), which allows a user to view “total patients”, “total claims”, and “total approved claims”, as well as year-to-date, month-to-date, and week-to-date statistics. FIG. 16B is an alternate preferred embodiment of the “Dashboard” interface for system 10, which includes a “task” submenu option to create tasks for specific users. The task option allows assignment of the task, due date, priority, description, and uploading of any necessary documents or attachments. A comment box is also provided on this screen.

The EHR system includes a messaging feature between the biller to clinic or clinician to clinician, so in the case the biller is from MMC or at the clinic itself, the biller can now message the clinician to update the date of birth, change insurance information with a new effective date, or message different departments for tasks needed in preparation of seeing the patient of while the patient is in front of the clinician. It is a messaging system to communicate from department to department.

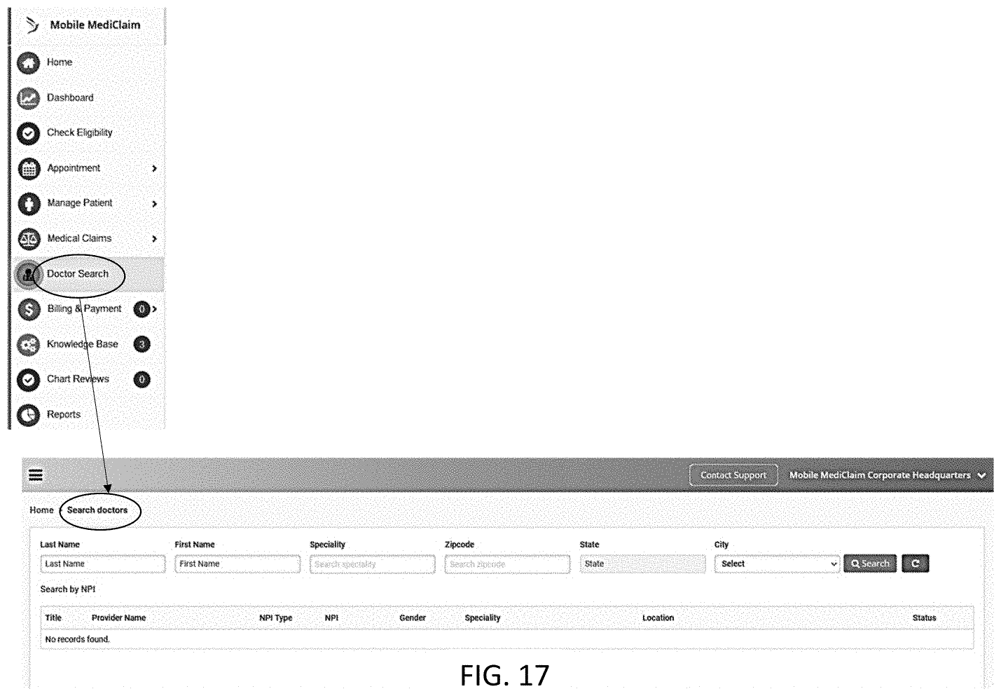

FIG. 17 is portions of a screen shot of the “Doctor Search” interface which allows physician searches by first/last name, specialty, or location (e.g., city, state, zip code).

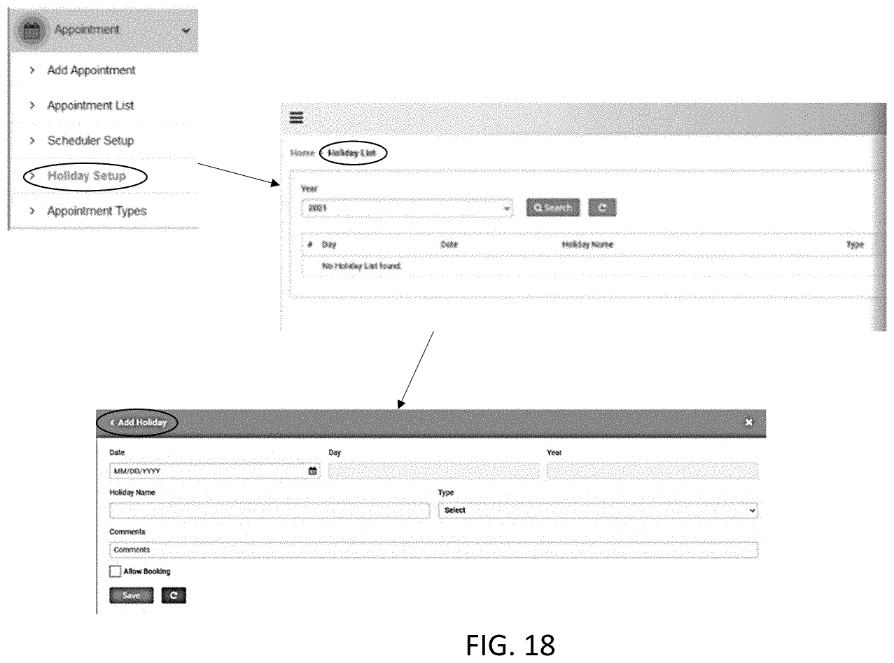

Under the “Appointment” interface menu option, FIG. 18 shows portions of a screen shot that allows a system administrator to set up and add holidays to the calendar under the submenu option of “Holiday setup.”

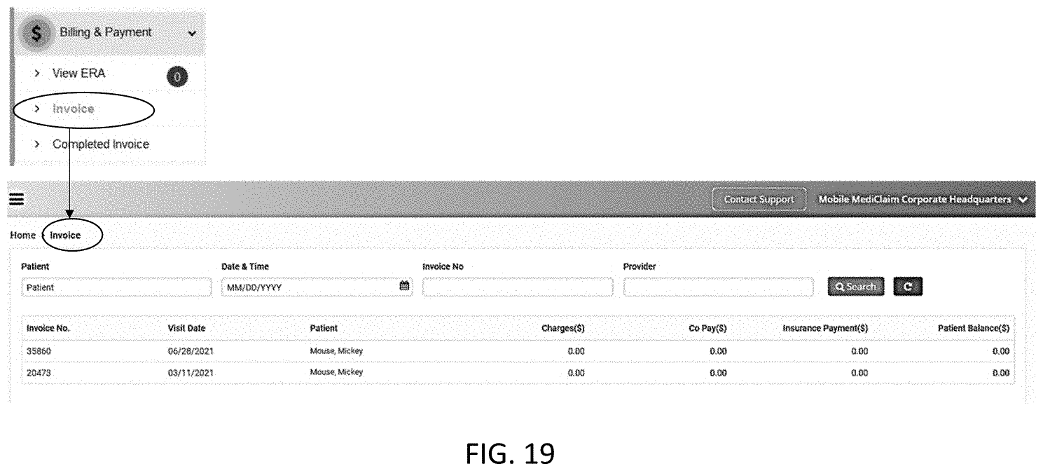

FIG. 19 is portions of a screen shot for the submenu option of “Invoice” under the “Billing & Payment” interface. This screen allows users to view, edit, and complete invoices for appointments. Searches or filters can be used based on patient name, date, invoice no., and insurance provider, with qualifying entries being listed.

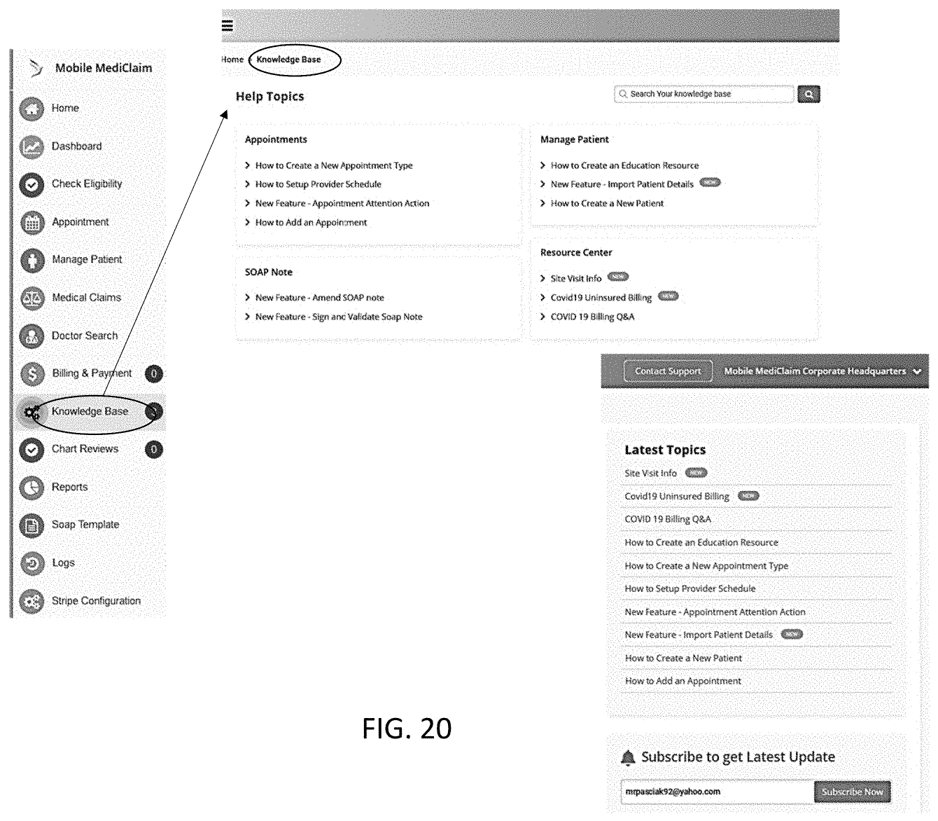

As a user interactive and learning tool, the system includes a “Knowledge Base” interface, as shown in FIG. 20. This feature gives users access to a knowledge base library with numerous help topics to facilitate navigation of the system and provide information on use and helpful tips. Users can subscribe to updates if they choose.

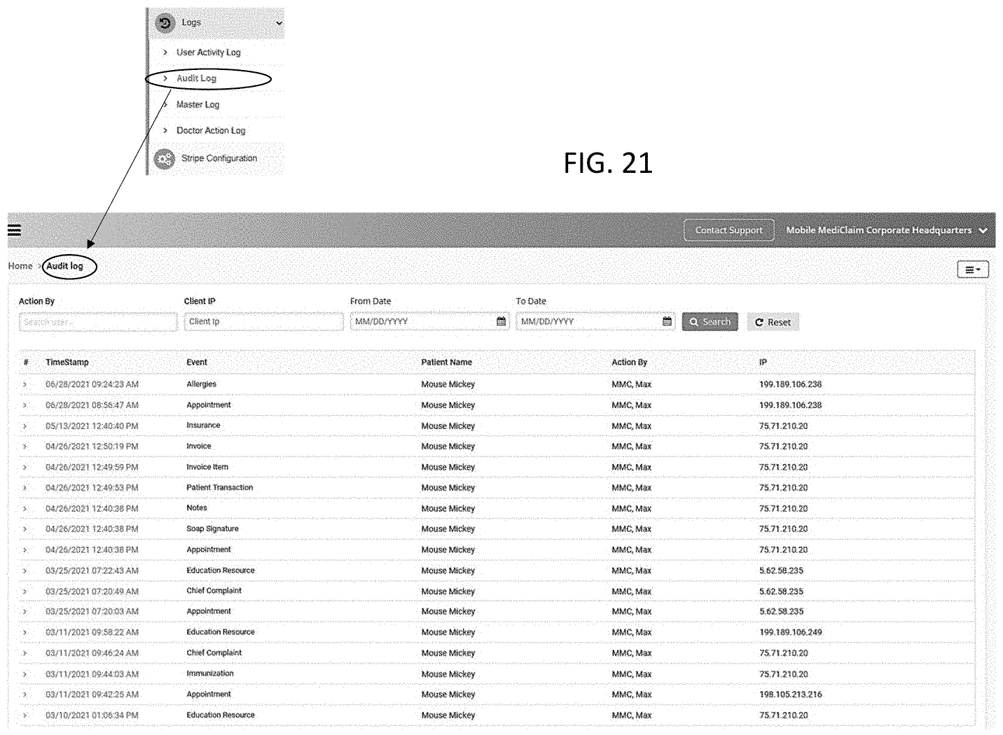

FIG. 21 is portions of a screen shot showing timestamped entries for each action in the system. The “events” can be searched by client name, action (or person taking action), or by a date range. Qualifying events will be listed with timestamp into the system 10.

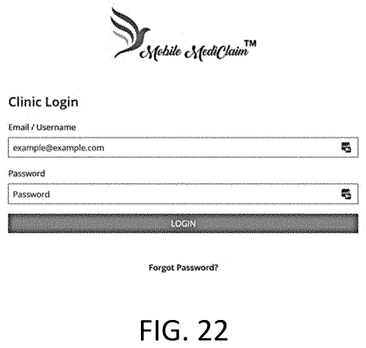

FIG. 22 is a screen shot of a login interface. Each user will have a personal login username and password. This allows administrators of the system to verify users and monitor user activity, if needed.

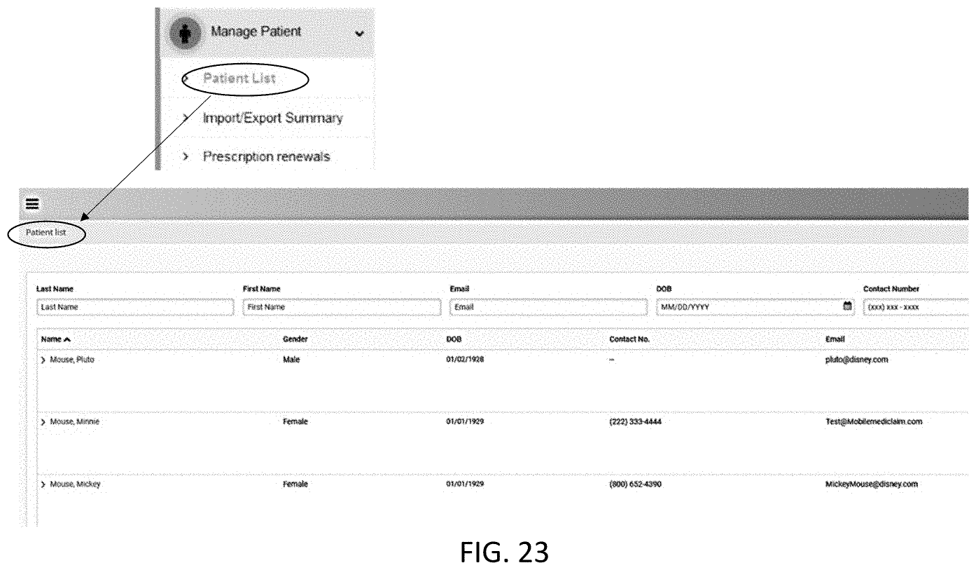

FIG. 23 is portions of a screen shot showing a submenu option “Patient list” under the “Manage Patient” interface. This option allows users to view, modify, or remove patients. Another feature of this interface allows importing patient information via .XML or .CSV.

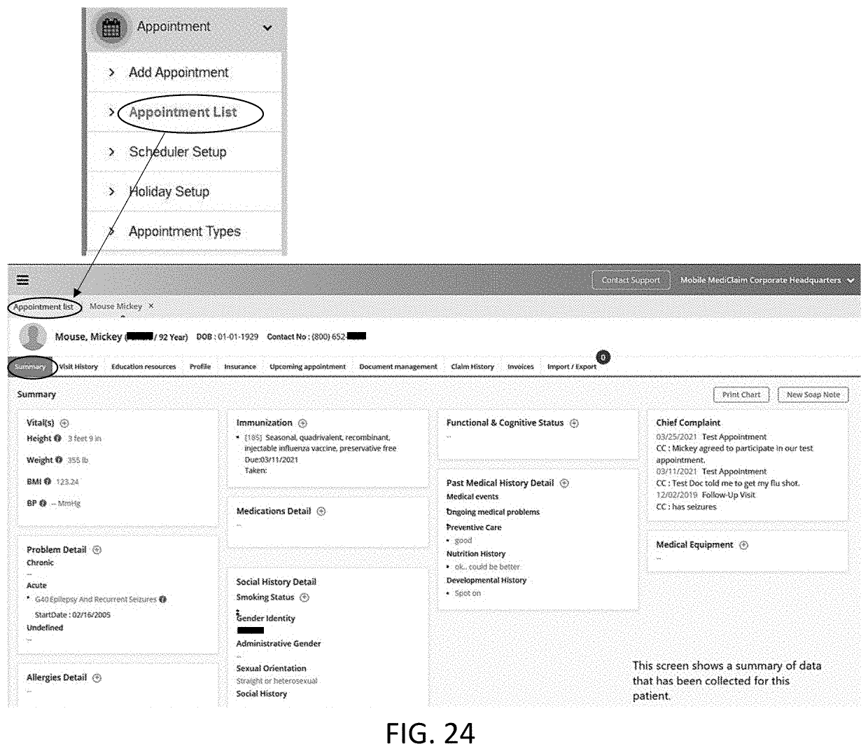

Under the menu interface option “Appointment” are several submenu options, including “Add Appointment,” “Appointment List,” “Scheduler Setup,” “Holiday Setup,” (see FIG. 18), and “Appointment Types.” FIG. 24 shows a screen having a summary of data, both medical and personal, collected for a specific selected patient.

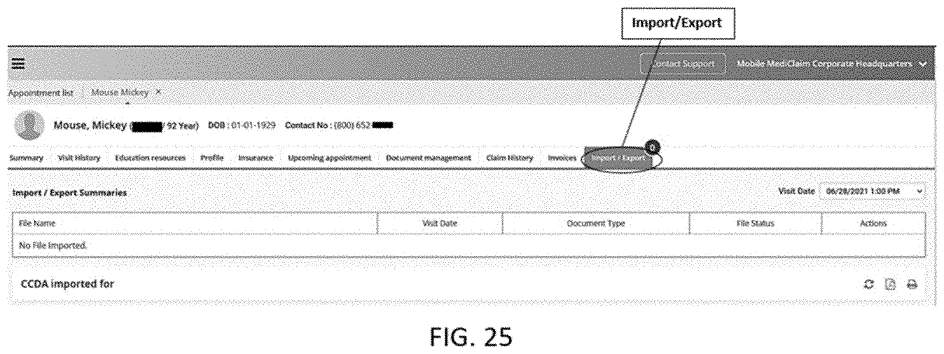

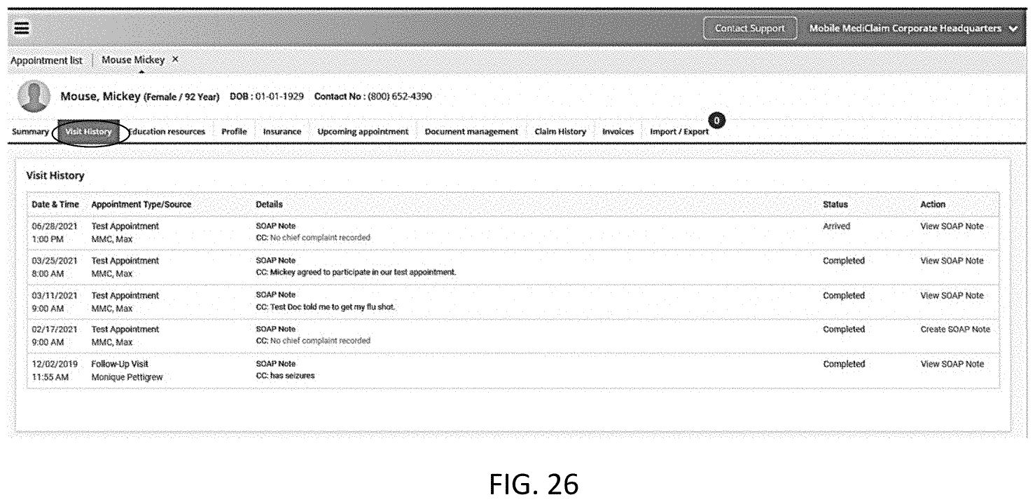

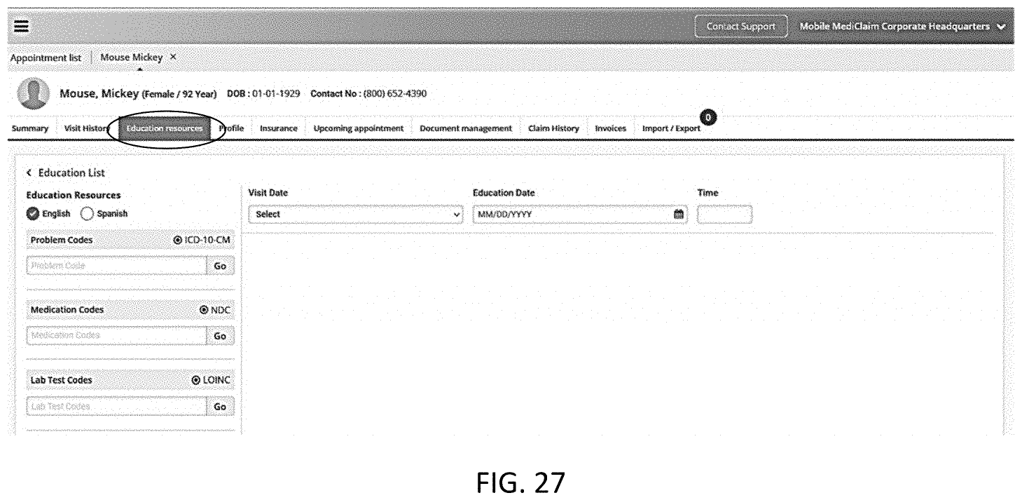

Using the selected patient (M. Mouse), and with further reference to related FIGS. 25-33, the screens navigated by selecting the tab options under the “Appointment List” option are shown. FIG. 25 is a partial screen shot showing an interface which allows a user to import and merge CCDA data received via HL7 with existing patient data in the system. FIG. 26 is a partial screen shot showing an interface option which allows a user to view appointment histories for the patient. Users can also view completed SOAP notes from this screen. FIG. 27 is a partial screen shot showing an interface which allows a user to create education entries with titles, dates, and links, as necessary. Users can search topics they wish to discuss and retrieve additional information on topics from the National Institute of Health (NIH). A user can start a timer to track time spent on educating patients. The education resources can even be selected for a language choice (e.g., English or Spanish).

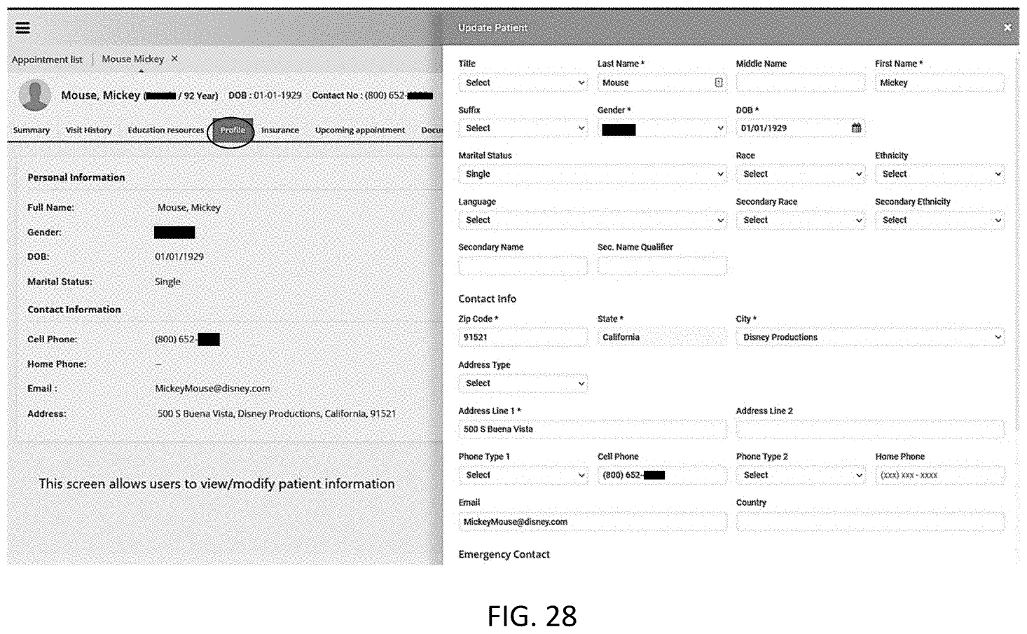

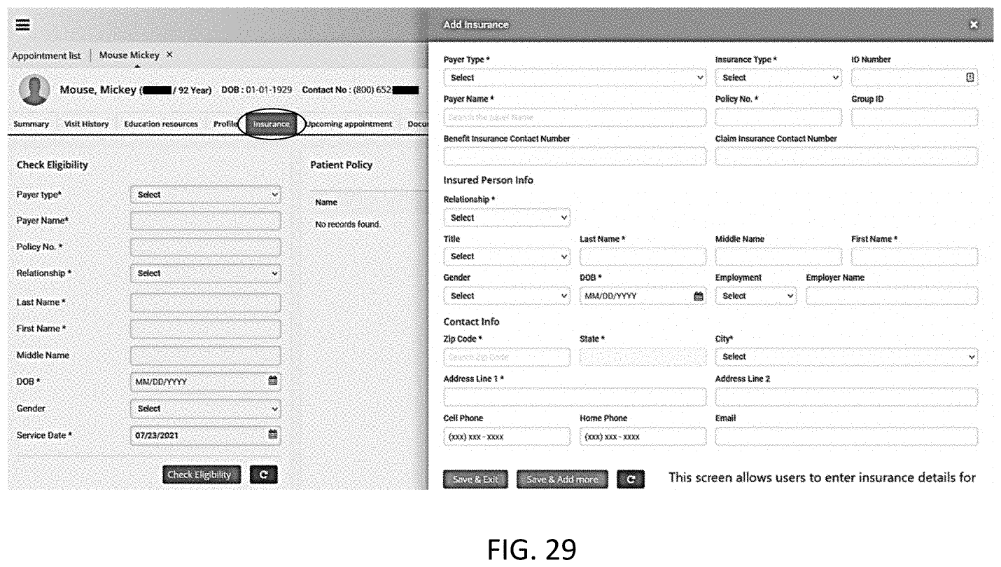

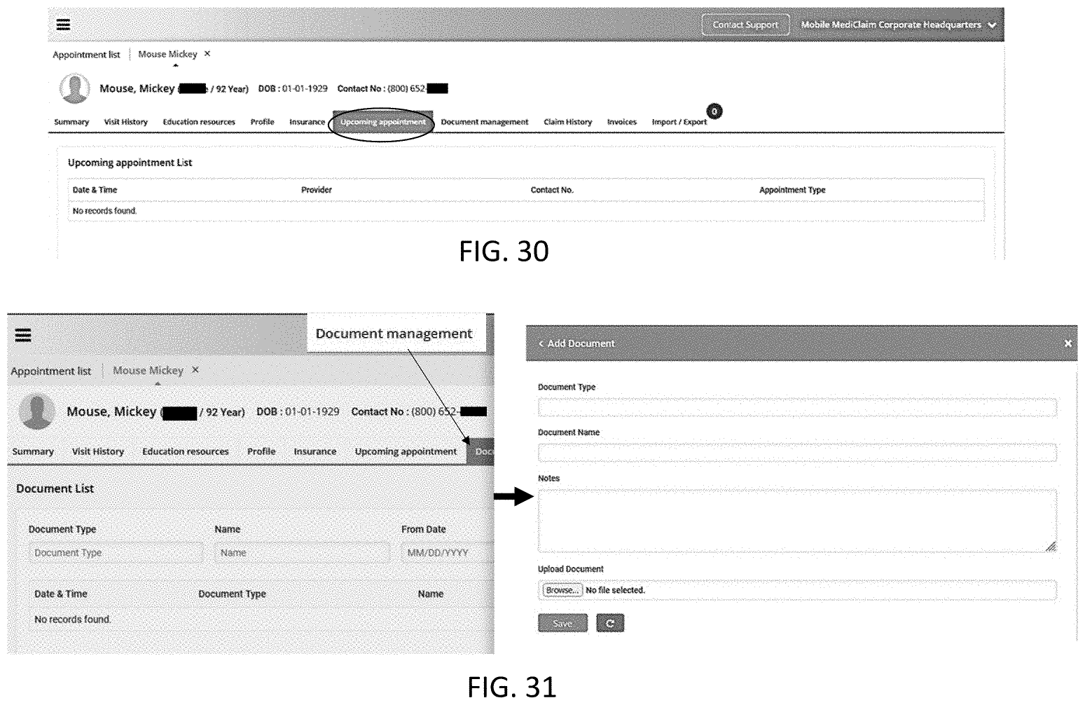

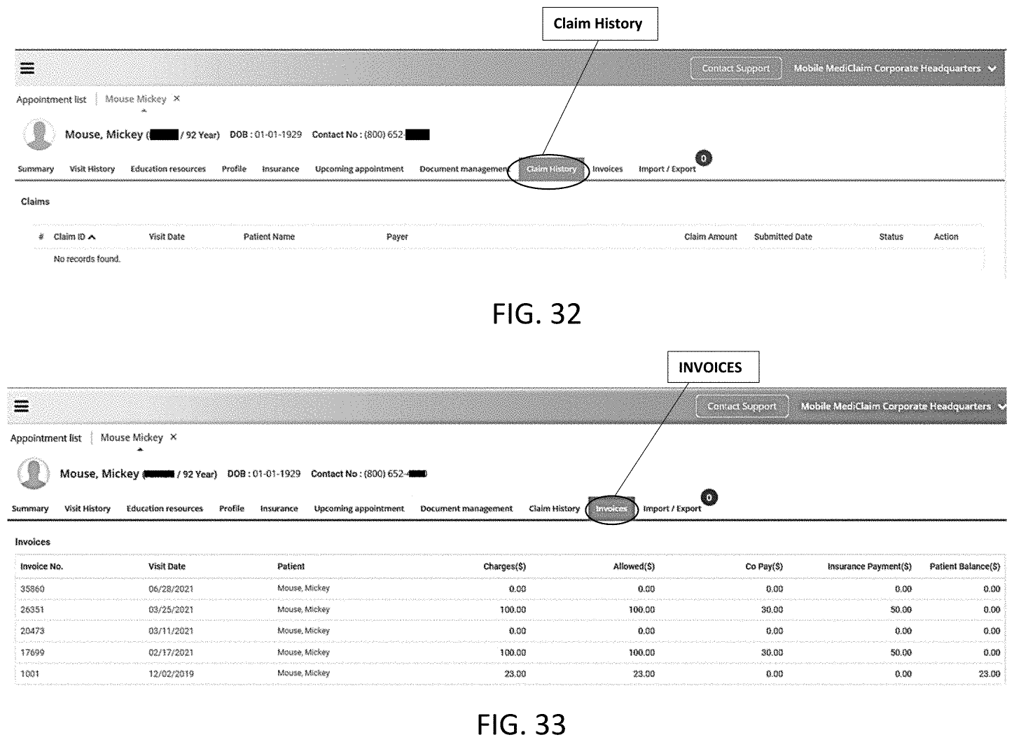

FIG. 28 is a double-paned screen shot showing the “Profile” tab option, which allows a user to view and modify patient personal information. The left pane shows information as it is currently in the system 10, while the right pane provides the ability to input additional or modify current information. FIG. 29 is another double-paned screen shot and shows the “Insurance” tab details. This interface allows a user to view and enter insurance details for the patient. FIG. 30 is a partial screen shot of the “Upcoming appointment” tab where a user can see the date, time and appointment type for future scheduled visits for the patient. FIG. 31 is two partial screen shots showing the “Document Management” tab of the “Appointment List” interface. In these screens a user can see documents for the patient and/or upload patient files to the system. FIG. 32 is a partial screen shot of the “Claim History” tab. This screen allows a user to see claims associated with the specific patient. Finally, under the “Appointment List” interface, FIG. 33 is a partial screen shot of the “Invoices” tab which provides a list of invoices associated with the patient.

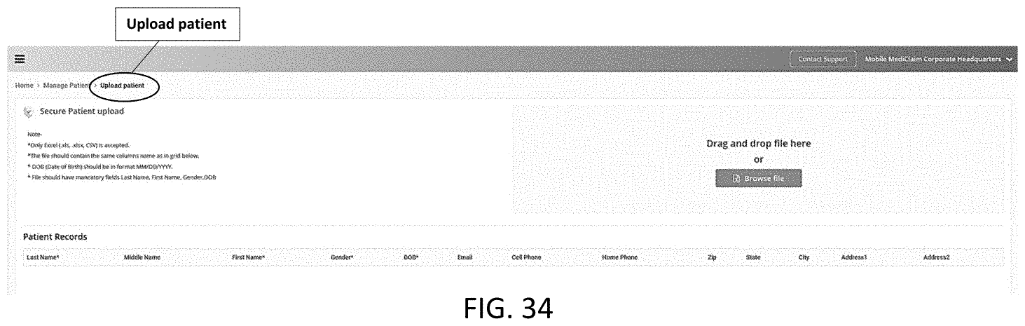

With reference to FIG. 34, a screen shot showing an “Upload Patient” option allows a user to upload patient data in .XML or .CSV.

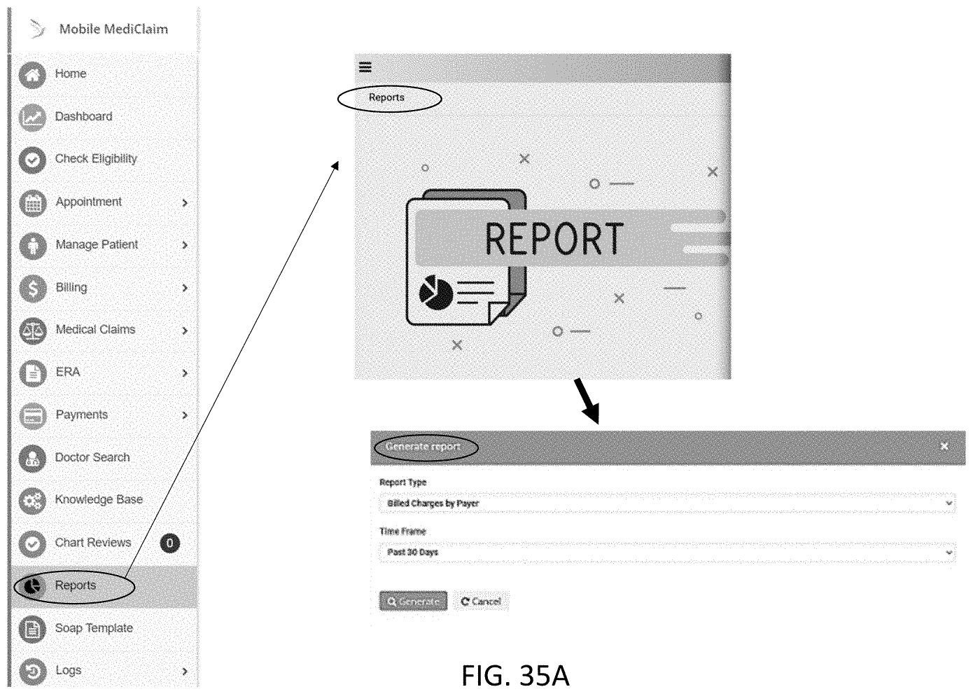

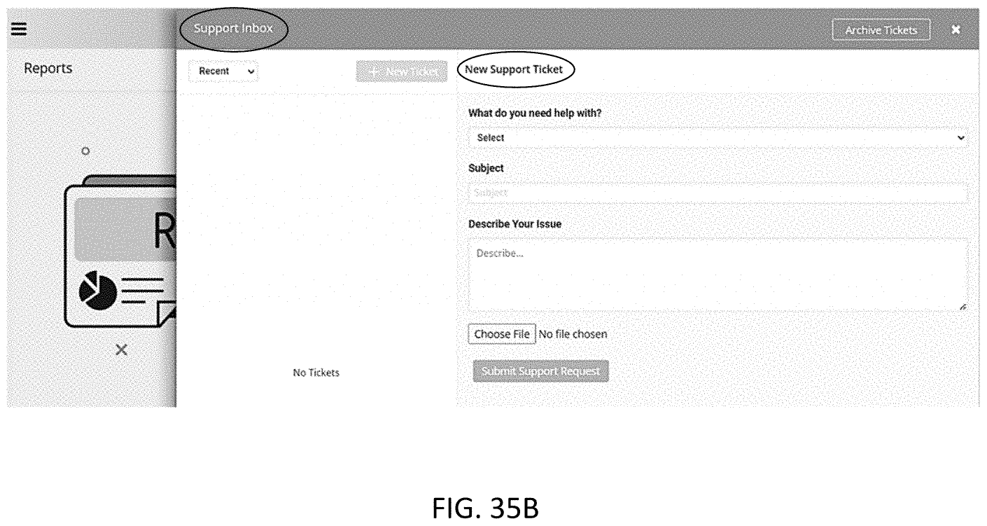

FIGS. 35A and 35B, are portions of a screen shots under the “Reports” menu interface. The screen of FIG. 35A allows a user to generate reports based on data in an account. The data for the report can be filtered and displayed based on the selected “report type” and the selected date range. FIG. 35B allows a user to communicate with support personnel when in need of assistance.

Reporting enhancements over prior systems involve all aspects of the Electronic Remittance Advice, ERA. The system has the ability to be report to the client. Reporting enhancements center around: rejections per CPT code, per diagnosis, per payor, per provider, date of service, any information that is shown in the ERA are now reportable.

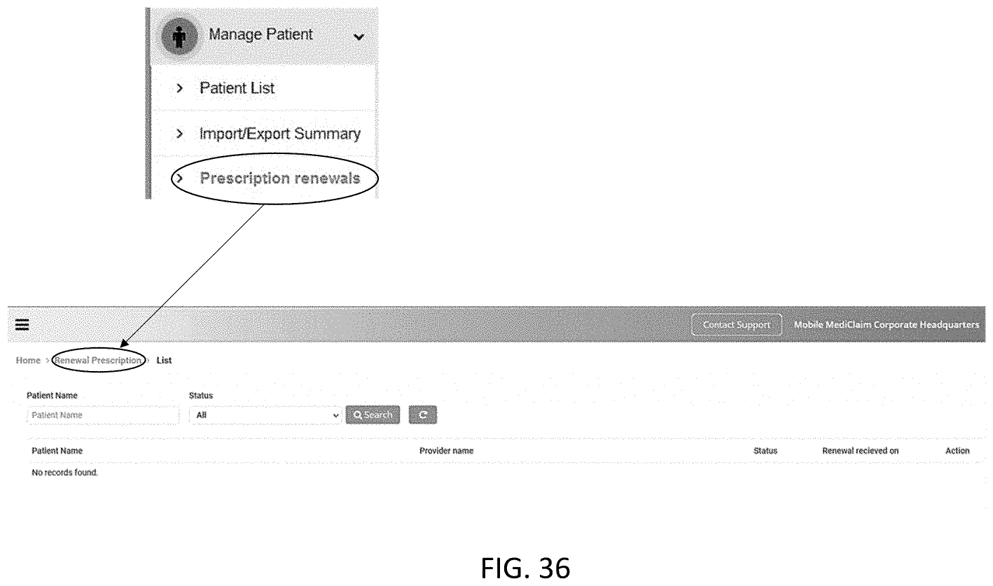

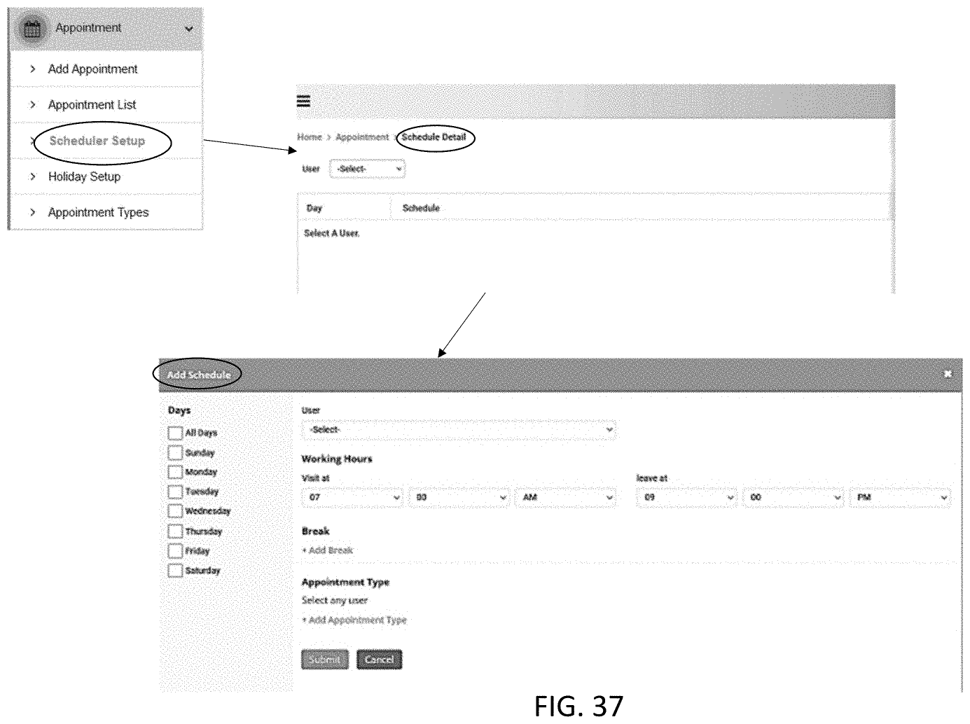

Returning to the “Manage Patient” interface menu option, FIG. 36 shows portions of a screen shot which allows a user to search and review prescription renewal requests from patients. FIG. 37 is a screen shot showing an interface which allows a user to view, create and modify weekly schedules for providers in a facility. Users also have the ability to associate appointment types when creating a schedule.

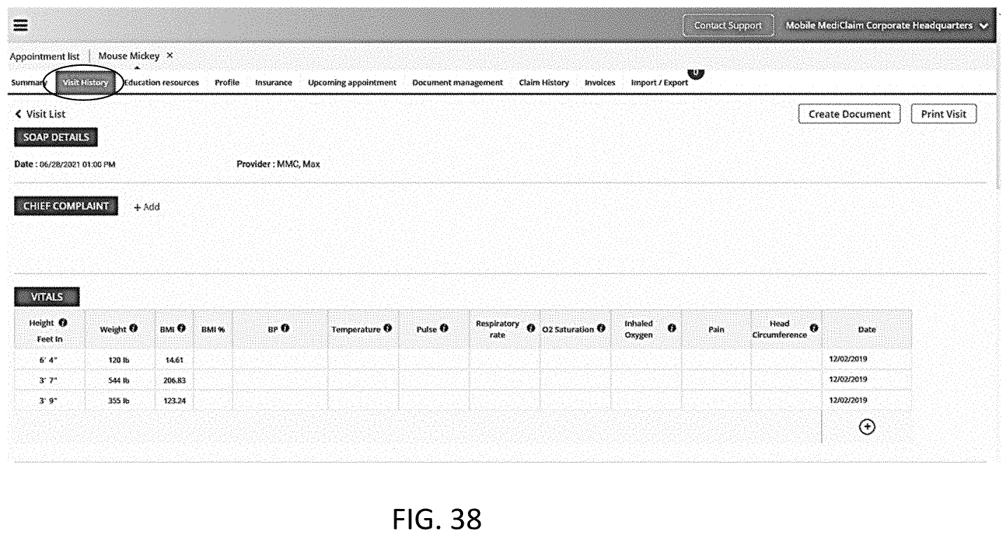

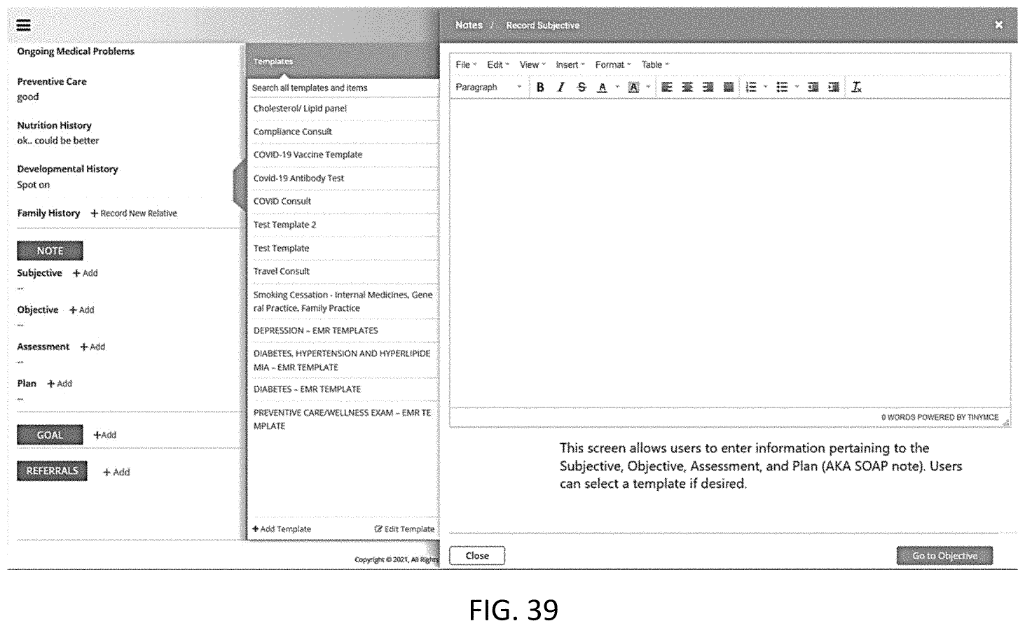

FIG. 36 is a screen shot showing an interface which allows a user to capture relevant information regarding a patient while conducting an appointment FIG. 37 is portions of a screen shot showing the “Scheduler Setup” option under the “Appointment” interface. The screens provide the user with an option to schedule employee work schedules. FIG. 38 is partial screen shot of the “View History” tab under the “Appointment” interface option. Note the “SOAP Details” option below the tab header. Selection of this option takes the user to FIG. 39.

FIG. 39 is the screen interface which allows a user to enter information pertaining to the Subjective, Objective, Assessment, and Plan (aka SOAP note). Various templates are available for users as well, as shown under the “Templates” option on the first pull-down submenu, including templates for diabetes and remote patient monitoring.

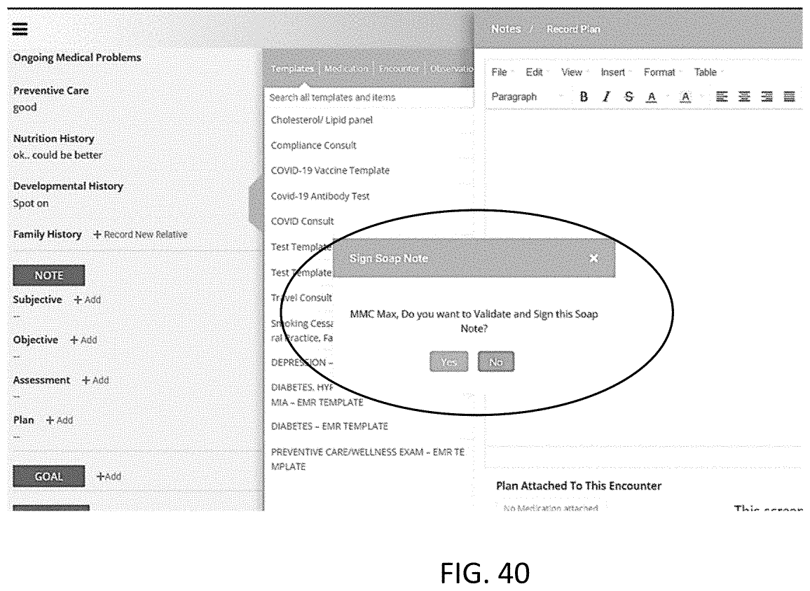

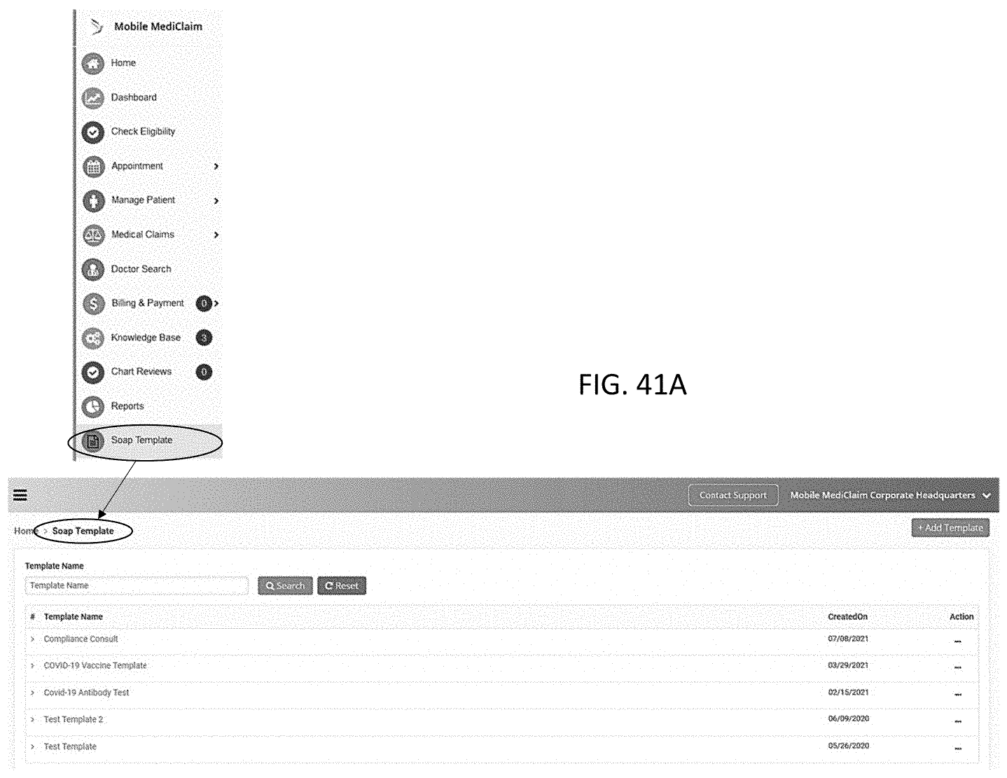

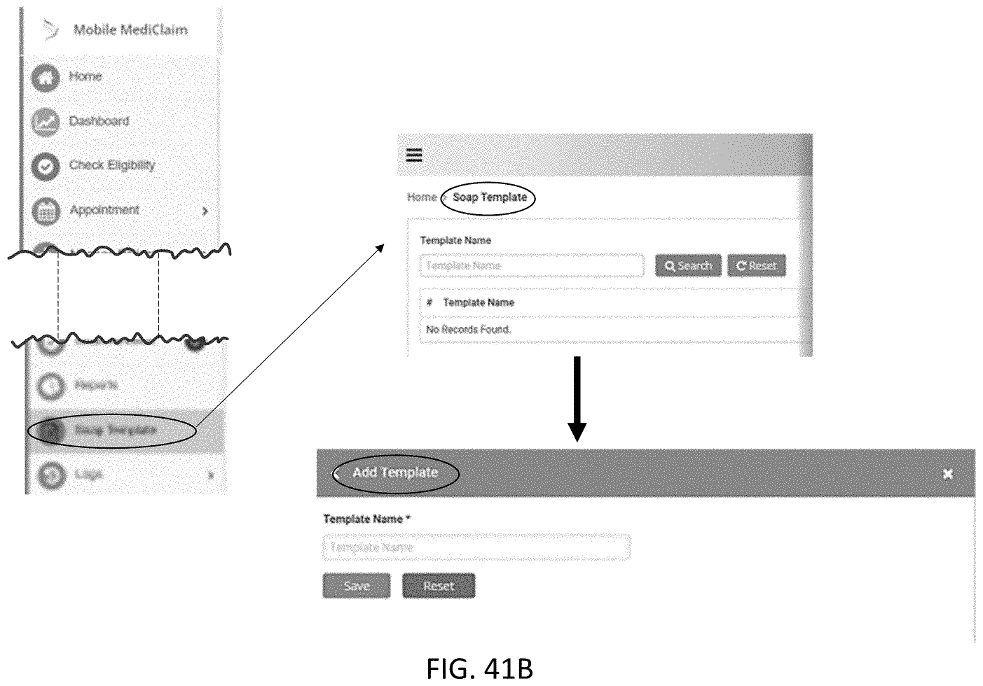

The SOAP details may be validated, as shown in FIG. 40, where a user can also sign the SOAP details by superimposing a signature onto the details once an appointment is complete. FIGS. 41A and 41B show two alternative submenu options under the “SOAP Template” interface which allows a user to create custom templates to be used in SOAP note creation for appointments and such.

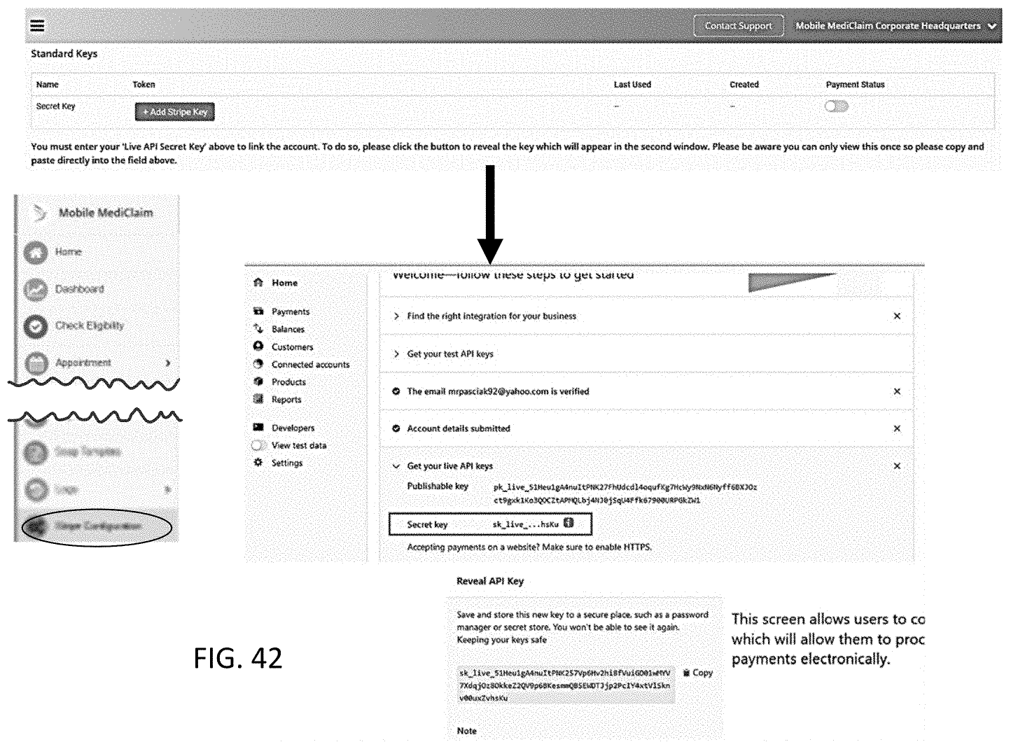

FIG. 42 is portions of a screen shot showing a “Stripe Configuration” interface which allows a user to configure a Stripe account for electronic processing and payment of invoices. Account transactions are encrypted for security purposes.

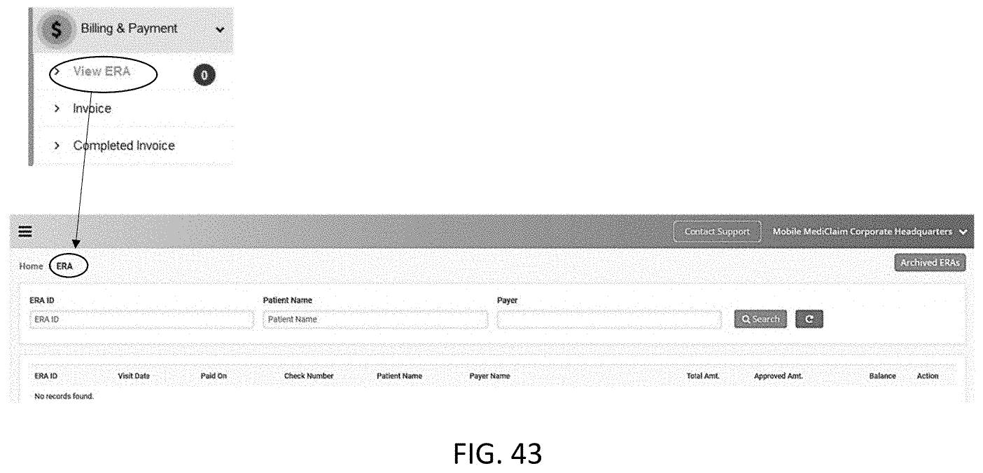

Finally, FIG. 43 is another embodiment for viewing ERA details, using the alternate menu under “Billing & Payment” interface and “View ERA” submenu option. The screen shot shows an interface which allows a user to review any Electronic Remittance Advice (ERA) documents that have been received from insurance carriers in response to claims. As previously noted, the Electronic Remittance Advice capabilities are fully integrated with Clearinghouse mirrors that reflect back into the system EHR.

The matter set forth in the foregoing description and accompanying drawings is offered by way of illustration only and not as a limitation. While particular embodiments have been shown and described, it will be apparent to those skilled in the art that changes and modifications may be made without departing from the broader aspects of applicants' contribution. The actual scope of the protection sought is intended to be defined in the following claims when viewed in their proper perspective based on the prior art.

What is claimed is:

1. A cloud-based medical billing and scheduling system comprising:
one or more network servers;
one or more portable electronic devices; and
an application downloadable onto the one or more portable electronic devices;
wherein the one or more portable electronic devices can send desired information from the application or receive desired information to and from the one or more network servers;
wherein the one or more portable electronic devices can display the desired information through the application.

2. The cloud-based medical system of claim 1, wherein the desired information includes patient information and medical claim information.

3. The cloud-based medical system of claim 1, wherein the application can display the desired information in a usable manner.

4. The cloud-based medical system of claim 1, wherein the application can receive information from an end user.

5. The cloud-based medical system of claim 1, wherein the application can run one or more diagnostic tests on the desired information.

6. The cloud-based medical system of claim 5, wherein the application can produce one or more graphs and one or more tables based on results curated from the diagnostic tests.

7. A method for providing a cloud-based medical system comprising:
receiving desired information through a computer application;
sending the desired information to one or more portable electronic devices;
sending the desired information from the one or more portable electronic devices to one or more network servers;
receiving the desired information from the one or more network servers to the one or more portable electronic devices;
displaying the desired information through the computer application in a usable manner.

8. The method of claim 7, further comprising running diagnostic tests on the desired information.

9. The method of claim 8, further comprising producing one or more graphs or one or more tables based on results curated from the diagnostic tests.
